(19) United States
(12) Patent Application Publication
(10) Pub. No.: US 2024/0240219 A1
(43) Pub. Date: Jul. 18, 2024

(54) METHODS OF ENRICHING FOR CIRCULAR POLYRIBONUCLEOTIDES

(71) Applicant: Flagship Pioneering Innovations VI, LLC, Cambridge, MA (US)

(72) Inventors: Randi Michelle KOTLAR, Arlington, MA (US); William Edward SALOMON, West Roxbury, MA (US)

(21) Appl. No.: 18/561,964

(22) PCT Filed: May 18, 2022
(86) PCT No.: PCT/US2022/029773

§ 371 Date: Nov. 17, 2023

Related U.S. Application Data
(60) Provisional application No. 63/190,001, filed on May 18, 2021.

(51) Int. Cl. C12P 19/34

(52) U.S. Cl. CPC C12P 19/34

(57) ABSTRACT

The methods of the disclosure can be used to enrich a population of circular polyribonucleotides in a mixture of linear polyribonucleotides, circular polyribonucleotides, and linear polydeoxyribonucleotides.

### SEQUENCE LISTING

The present application is being filed along with a Sequence Listing in electronic format. The Sequence Listing is provided as a file entitled 51509-041WO2_Sequence_Listing_5_16_22_ST25 created on May 16, 2022, which is 5,358 bytes in size. The information in electronic format of the sequence listing is incorporated herein by reference in its entirety.

### BACKGROUND OF THE INVENTION

Circular polyribonucleotides show increased resistance to degradation by nucleases resulting in a longer half-life in comparison to linear polyribonucleotides. Circular polyribonucleotides are known to occur endogenously or may be circularized exogenously. The exogenous circularization reaction results in a mixture of successfully circularized polyribonucleotides in addition to some residual linear polyribonucleotides. The presence of linear polyribonucleotides in pharmaceutical circular polyribonucleotide preparations can have unexpected and undesired effects. Thus, there remains a need for methods of enriching, separating, and/or purifying circular polyribonucleotides relative to the linear polyribonucleotides.

### SUMMARY OF THE INVENTION

This disclosure provides methods of producing an enriched population of circular polyribonucleotides. In particular, the disclosure provides methods of producing an enriched population of circular polyribonucleotides from a mixture of linear polyribonucleotides, circular polyribonucleotides, and linear polydeoxyribonucleotides by digesting linear polydeoxyribonucleotide with DNase I followed by digesting the linear polyribonucleotides with a 5′ exonuclease or a 3′ exonuclease.

In an aspect, the disclosure provides a method of producing an enriched population of circular polyribonucleotides including providing a splint ligation reaction product comprising circular polyribonucleotides, linear polyribonucleotides, and linear polydeoxyribonucleotides; reacting the splint ligation reaction product with DNase I, wherein Dnase I digests at least a portion of the linear polydeoxyribonucleotides to produce a first digested mixture; and reacting the first digested mixture with an exonuclease, wherein the exonuclease digests at least a portion of the linear polyribonucleotides to produce a second digested mixture; wherein the second digested mixture comprises an enriched population of circular polyribonucleotides. In some embodiments, the Dnase I is in an amount of between 0.1 U/μg to 1 U/μg (e.g., between 0.1 U/μg and 0.8 U/μg, between 0.1 U/μg and 0.6 U/μg, between 0.1 U/μg and 0.4 U/μg, between 0.1 U/μg and 0.2 U/μg, between 0.2 U/μg and 1 U/μg, between 0.4 U/μg and 1 U/μg, between 0.6 U/μg and 1 U/μg, or between 0.8 U/μg and 1 U/μg).

In some embodiments, the Dnase I digesting step is performed for at least 10 minutes (e.g., at least 15 minutes, at least 30 minutes, at least 1 hour, at least 2 hours, at least 12 hours, and at least 24 hours). In some embodiments, the Dnase I digesting step is performed at a temperature of about 37° C.

In some embodiments, the exonuclease that digests at least a portion of the linear polyribonucleotides to produce a second digested mixture is a 5′ exonuclease. In some embodiments, the 5′ exonuclease which digests at least a portion of the linear polyribonucleotides to produce a second digested mixture is a 5′-phosphate dependent exonuclease. In some embodiments, the 5′ exonuclease is Xm-1. In some embodiments, the Xrn-1 is in an amount of between 0.1 U/μg and 1 U/μg (e.g., between 0.1 U/μg and 0.8 U/μg, between 0.1 U/μg and 0.6 U/μg, between 0.1 U/μg and 0.4 U/μg, between 0.1 U/μg and 0.2 U/μg, between 0.2 U/μg and 1 U/μg, between 0.4 U/μg and 1 U/μg, between 0.6 U/μg and 1 U/μg, or between 0.8 U/μg and 1 U/μg).

In some embodiments, the exonuclease that digests at least a portion of the linear polyribonucleotides to produce a second digested mixture is a 3′ exonuclease. In some embodiments, the 3′ exonuclease is Exonuclease T.

In some embodiments, the digesting step, wherein the exonuclease digests at least a portion of the linear polyribonucleotides to produce a second digested mixture, is performed for at least 1 hour (e.g., at least 90 minutes, at least 2 hours, at least 6 hours, at least 12 hours, and at least 24 hours). In some embodiments, the digesting step, wherein the exonuclease digests at least a portion of the linear polyribonucleotides to produce a second digested mixture, is performed at a temperature of about 37° C.

In another aspect, the disclosure provides a method of producing an enriched population of circular polyribonucleotides including providing a linear polyribonucleotide having a 5′ and 3′ end and a polydeoxyribonucleotide that has a first region that hybridizes to the 5′ end of the linear polyribonucleotide and a second region that hybridizes to the 3′ end of the linear polyribonucleotide, ligating the 5′ end of the linear polyribonucleotide to the 3′ end of the linear polyribonucleotide to produce a splint ligation reaction product comprising circular polyribonucleotides, linear polyribonucleotides, and linear polydeoxyribonucleotides; reacting the splint ligation reaction product with Dnase I, wherein the Dnase I digests at least a portion of the polydeoxyribonucleotide to produce a first digested mixture; reacting the first digested mixture with an exonuclease, wherein the exonuclease digests at least a portion of the linear polyribonucleotide to produce a second digested mixture, wherein the second digested mixture comprises an enriched population of circular polyribonucleotides.

In some embodiments, the Dnase I which digests at least a portion of the linear polydeoxyribonucleotides to produce a first digested mixture is in an amount of between 0.1 U/μg and 1 U/μg (e.g., between 0.1 U/μg and 0.8 U/μg, between 0.1 U/μg and 0.6 U/μg, between 0.1 U/μg and 0.4 U/μg, between 0.1 U/μg and 0.2 U/μg, between 0.2 U/μg and 1 U/μg, between 0.4 U/μg and 1 U/μg, between 0.6 U/μg and 1 U/μg, or between 0.8 U/μg and 1 U/μg). In some embodiments, the digesting step of DNase I digesting at least a portion of the linear polydeoxyribonucleotides to produce a first digested mixture is performed for at least 10 minutes (e.g., at least 15 minutes, at least 30 minutes, at least 1 hour, at least 2 hours, at least 12 hours, and at least 24 hours). In some embodiments, the digesting step of DNase I digesting at least a portion of the linear polydeoxyribonucleotides to produce a first digested mixture is performed at a temperature of about 37° C.

In some embodiments, the exonuclease that digests at least a portion of the linear polyribonucleotides to produce a second digested mixture is a 5′ exonuclease. In some embodiments, the 5′ exonuclease which digests at least a portion of the linear polyribonucleotides to produce a second digested mixture is a 5′-phosphate dependent exonuclease. In some embodiments, the 5′ exonuclease is Xm-1. In some embodiments, the Xrn-1 is in an amount of between 0.1 U/μg and 1 U/μg (e.g., between 0.1 U/μg and 0.8 U/μg, between 0.1 U/μg and 0.6 U/μg, between 0.1 U/μg and 0.4 U/μg, between 0.1 U/μg and 0.2 U/μg, between 0.2 U/μg and 1 U/μg, between 0.4 U/μg and 1 U/μg, between 0.6 U/μg and 1 U/μg, or between 0.8 U/μg and 1 U/μg). In some embodiments, the exonuclease that digests at least a portion of the linear polyribonucleotides to produce a second digested mixture is a 3′ exonuclease. In some embodiments, the 3′ exonuclease is Exonuclease T. In some embodiments, the digesting step, wherein the exonuclease digests at least a portion of the linear polyribonucleotides to produce a second digested mixture, is performed for at least 1 hour (e.g., at least 90 minutes, at least 2 hours, at least 6 hours, at least 12 hours, and at least 24 hours). In some embodiments, the digesting step, wherein the exonuclease digests at least a portion of the linear polyribonucleotides to produce a second digested mixture, is performed at a temperature of about 37° C.

### Definitions

To facilitate the understanding of this invention, a number of terms are defined below. Terms defined herein have meanings as commonly understood by a person of ordinary skill in the areas relevant to the invention. Terms such as “a,”, “an,” and “the” are not intended to refer to only a singular entity, but include the general class of which a specific example may be used for illustration. The terminology herein is used to describe specific embodiments of the invention, but their usage does not limit the invention, except as outlined in the claims.

As used herein, any values provided in a range of values include both the upper and lower bounds, and any values contained within the upper and lower bounds.

As used herein, the term “3′ exonuclease” refers to an enzyme having exonuclease activity wherein the enzyme removes nucleotides from a strand of nucleotides using hydrolysis starting from the 3′ end of the strand of nucleotides and continuing in a processive manner toward the 5′ end of the strand of nucleotides. 3′ exonucleases include but are not limited to Exonuclease T, Polynucleotide Phosphorylase, RNase D, RNase R, and Exoribonuclease II.

As used herein, the term “5′ exonuclease” refers to an enzyme having exonuclease activity wherein the enzyme removes nucleotides from a strand of nucleotides using hydrolysis starting from the 5′ end of the strand of nucleotides and continuing in a processive manner toward the 3′ end of the strand of nucleotides. 5′ exonucleases include not are not limited to Xrn-1, A Exonuclease, T7 Exonuclease, Exonuclease VII, and Terminator™.

As used herein, the term “5′-phosphate dependent exonuclease” refers to an exonuclease that digests polynucleotides having a 5′ monophosphate in a 5′-to-3′ processive manner (e.g., Terminator™ and Xrn-1).

As used herein, the terms “circRNA,” “circular polyribonucleotide,” “circular RNA,” and “circular polyribonucleotide molecule” are used interchangeably and mean a polyribonucleotide molecule that has a structure having no free ends (i.e., no free 3′ or 5′ end), for example a polyribonucleotide molecule that forms a circular or end-less structure through covalent or non-covalent bonds.

As used herein, the term “circularization efficiency” is a measurement of resultant circular polyribonucleotide versus its non-circular starting material.

As used herein, the terms “circRNA preparation,” “circular polyribonucleotide preparation,” and “circular RNA preparation” are used interchangeably and mean a composition including circRNA molecules and a diluent, carrier, first adjuvant, or a combination thereof.

As used herein, the term “digested mixture” refers to a mixture including linear polyribonucleotides, circular polyribonucleotides, and optionally linear polydeoxyribonucleotides that is produced by contacting a mixture of linear polyribonucleotides, circular polyribonucleotides, and optionally linear polydeoxyribonucleotides with a digesting enzyme (e.g., DNase I or an exonuclease).

As used herein, the term “enriched population” refers to a population of polyribonucleotides that has a higher percentage of circular polyribonucleotides in comparison to another population of circular and linear polyribonucleotides.

As used herein, the terms “fragment” and “portion” mean any part of a polynucleotide molecule that is at least one nucleotide shorter than the polynucleotide molecule. For example, a nucleotide molecule can be a linear polyribonucleotide molecule and a fragment thereof can be a monoribonucleotide or any number of contiguous polyribonucleotides that are a portion of the linear polyribonucleotide molecule.

As used herein, the term “impurity” is an undesired substance present in a composition, e.g., a pharmaceutical composition as described herein. In some embodiments, an impurity is a process-related impurity. In some embodiments, an impurity is a product-related substance other than the desired product in the final composition, e.g., other than the active drug ingredient, e.g., circular, or linear polyribonucleotide, as described herein. As used herein, the term “process-related impurity” is a substance used, present, or generated in the manufacturing of a composition, preparation, or product that is undesired in the final composition, preparation, or product other than the linear polyribonucleotides described herein. In some embodiments, the process-related impurity is an enzyme used in the synthesis or circularization of polyribonucleotides. As used herein, the term “product-related substance” is a substance or byproduct produced during the synthesis of a composition, preparation, or product, or any intermediate thereof. In some embodiments, the product-related substance is deoxyribonucleotide fragments. In some embodiments, the product-related substance is deoxyribonucleotide monomers. In some embodiments, the product-related substance is one or more of: derivatives or fragments of polyribonucleotides described herein, e.g., fragments of 10, 9, 8, 7, 6, 5, or 4 ribonucleic acids, monoribonucleic acids, diribonucleic acids, or triribonucleic acids.

As used herein, the term “linear counterpart” is a polyribonucleotide molecule (and its fragments) having the same or similar nucleotide sequence (e.g., 100%, 95%, 90%, 85%, 80%, 75%, or any percentage therebetween sequence identity) as a circular polyribonucleotide and having two free ends (i.e., the uncircularized version (and its fragments) of the circularized polyribonucleotide). In some embodiments, the linear counterpart (e.g., a pre-circularized version) is a polyribonucleotide molecule (and its fragments) having the same or similar nucleotide sequence (e.g., 100%, 95%, 90%, 85%, 80%, 75%, or any percentage therebetween sequence identity) and same or similar nucleic acid modifications as a circular polyribonucleotide and having two free ends (i.e., the uncircularized version (and its fragments) of the circularized polyribonucleotide). In some embodiments, the linear counterpart is a polyribonucleotide molecule (and its fragments) having the same or similar nucleotide sequence (e.g., 100%, 95%, 90%, 85%, 80%, 75%, or any percentage therebetween sequence identity) and different or no nucleic acid modifications as a circular polyribonucleotide and having two free ends (i.e., the uncircularized version (and its fragments) of the circularized polyribonucleotide). In some embodiments, a fragment of the polyribonucleotide molecule that is the linear counterpart is any portion of linear counterpart polyribonucleotide molecule that is shorter than the linear counterpart polyribonucleotide molecule. In some embodiments, the linear counterpart further includes a 5′ cap. In some embodiments, the linear counterpart further includes a poly adenosine tail. In some embodiments, the linear counterpart further includes a 3′ UTR. In some embodiments, the linear counterpart further includes a 5′ UTR.

As used herein, the terms “linear RNA,” “linear polyribonucleotide,” and “linear polyribonucleotide molecule” are used interchangeably and mean polyribonucleotide molecule having a 5′ and 3′ end. One or both of the 5′ and 3′ ends may be free ends or joined to another moiety. Linear RNA includes RNA that has not undergone circularization (e.g., is pre-circularized) and can be used as a starting material for circularization through, for example, splint ligation, or chemical, enzymatic, ribozyme- or splicing-catalyzed circularization methods.

As used herein, the term “mixture” means a material made of two or more different substances that are mixed. In some cases, a mixture described herein can be a homogenous mixture of the two or more different substances, e.g., the mixture can have the same proportions of its components (e.g., the two or more substances) throughout any given sample of the mixture. In some cases, a mixture as provided herein can be a heterogeneous mixture of the two or more different substances, e.g., the proportions of the components of the mixture (e.g., the two or more substances) can vary throughout the mixture. In some cases, the mixture includes circular polyribonucleotides and linear polyribonucleotides. In some embodiments, the mixture includes circular polyribonucleotides, linear polyribonucleotides, and linear polydeoxyribonucleotides. In some cases, a mixture is a liquid solution, e.g., the mixture is present in liquid phase. In some instances, a liquid solution can be regarded as comprising a liquid solvent and a solute. Mixing a solute in a liquid solvent can be termed as “dissolution” process. In some cases, a liquid solution is a liquid-in-liquid solution (e.g., a liquid solute dissolved in a liquid solvent), a solid-in-liquid solution (e.g., a solid solute dissolved in a liquid solvent), or a gas-in-liquid solution (e.g., a solid solute dissolved in a liquid solvent). In some cases, there is more than one solvent and/or more than one solute. In some cases, a mixture is a colloid, liquid suspension, or emulsion. In some cases, a mixture is a solid mixture, e.g., the mixture is present in solid phase.

As used herein, the term “modified ribonucleotide” means a nucleotide with at least one modification to the sugar, the nucleobase, or the internucleoside linkage.

The term “polynucleotide” as used herein means a molecule comprising one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Polyribonucleotides or ribonucleic acids, or RNA, can refer to macromolecules that include multiple ribonucleotides that are polymerized via phosphodiester bonds. Deoxyribonucleotides are nucleotides in which the sugar is deoxyribose.

Polydeoxyribonucleotides or deoxyribonucleic acids, or DNA, means macromolecules that include multiple deoxyribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, that include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a polynucleotide is deoxyribonucleic acid (DNA), ribonucleic acid (RNA), or derivatives or variants thereof. In some cases, a polynucleotide is a short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), to name a few, and encompasses both the nucleotide sequence and any structural embodiments thereof, such as single-stranded, double-stranded, triple-stranded, helical, hairpin, etc. In some cases, a polynucleotide molecule is circular. A polynucleotide can have various lengths. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. As embodied herein, the polynucleotide sequences may include isolated and purified DNA/RNA molecules, synthetic DNA/RNA molecules, and synthetic DNA/RNA analogs.

Polynucleotides, e.g., polyribonucleotides or polydeoxyribonucleotides, may include one or more nucleotide variants, including nonstandard nucleotide(s), non-natural nucleotide(s), nucleotide analog(s) and/or modified nucleotides. Modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as amino ally 1-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A, Lavergne T, Welte W, Diederichs K, Dwyer T J, Ordoukhanian P, Romesberg F E, Marx A. Nat. Chem. Biol. 2012 July; 8(7):612-4, which is herein incorporated by reference for all purposes.

As used herein, the phrase “quasi-helical structure” is a higher order structure of the circular polyribonucleotide, wherein at least a portion of the circular polyribonucleotide folds into a helical structure.

As used herein, the term “splint ligation reaction product” refers to the composition including circular polyribonucleotides, linear polyribonucleotides, and linear polydeoxyribonucleotides that is produced as a result of providing a linear polyribonucleotide having a 5′ and 3′ end and a polydeoxyribonucleotide that has a first region that hybridizes to the 5′ end of the linear polyribonucleotide and a second region that hybridizes to the 3′ end of the linear polyribonucleotide, and ligating the 5′ end of the linear polyribonucleotide to the 3′ end of the linear polyribonucleotide.

As used herein, the terms “total ribonucleotide molecules” and “total polyribonucleotides” mean the total amount of any ribonucleotide molecules, including linear polyribonucleotide molecules, circular polyribonucleotide molecules, monomeric ribonucleotides, other polyribonucleotide molecules, fragments thereof, and modified variations thereof, as measured by total mass of the ribonucleotide molecules.

As used herein, the term “unit” refers to the amount of enzyme required to perform a defined catalytic activity under specified methods of the assay method which are summarized for each enzyme in Table 1.

TABLE 1
Definitions of a unit for various enzymes

| Enzyme | One unit (U) is equal to the amount of enzyme that: |
|---|---|
| Xrn-1 | digests 1 μg of phosphorylated yeast RNA in 60 |
| | minutes at 37° C. in 100 mM NaCl, 50 mM Tris-HCl, 10 |
| | mM MgCl₂ and 1 mM dithiothreitol with a pH of 7.9 at |
| | 25° C. |
| RNase R | converts 1 μg of poly-r(A) into acid-soluble nucleotides |
| | in 10 minutes at 37° C. in 20 mM Tris-HCl (pH 8.0), 100 |
| | mM KCl and 0.1 mM MgCl₂. |
| Terminator ™ | digests 1 μg of rRNA substrate into acid-soluble |
| | nucleotides in 60 minutes at 30° C. under standard |
| | assay conditions. |
| Exonuclease T | produces 0.1 nmol of trichloroacetic acetic acid (TCA) |
| | soluble DNA from 1 nmol of [3H]-labeled polythymidine |
| | in 30 minutes at 25° C. in a total reaction volume of 100 |
| | μl in 50 mM potassium acetate, 20 mM Tris-acetate, 10 |
| | mM magnesium acetate, and 1 mM dithiothreitol with |
| | pH of 7.9 at 25° C. |
| Polynucleotide | polymerizes 1 μmol of ADP, releasing 1 μmol of |
| phosphorylase | inorganic phosphate in 15 minutes, at pH 9.1 at 37° C. |
| | in a solution of 20 mM HEPES buffer pH 7.9, 0.1 mM |
| | EDTA, 2 mM DTT, 12.5 mM MgCl2, ~130 mM KCl, |
| | and 20% (w/v) glycerol. |
| DNase I | degrades 1 μg of pBR322 DNA in 10 minutes at 37° C. |
| | in 10 mM Tris-HCl, 2.5 mM MgCl₂, 0.5 mM CaCl₂, at a |
| | pH of 7.6 at 25° C. |
| Lambda | produces 10 nmol of acid-soluble deoxyribonucleotide |
| Exonuclease | from double-stranded substrate in a total reaction |
| | volume of 50 μL in 30 minutes at 37° C. in buffer of 67 |
| | mM glycine-KOH, 2.5 mM MgCl2, 50 μg/ml BSA, with |
| | a pH of 9.4 at 25° C. with 1 μg sonicated duplex [3H]- |
| | DNA. |
| Exonuclease | results in the acid-solubilization of 1 nmol of |
| VII | nucleotides from activated single-stranded calf thymus |
| | DNA in 30 minutes at 37° C. in 50 mM Tris-HCl (pH |
| | 7.9), 50 mM sodium phosphate (pH 7.8), 2 mM 2- |
| | mercaptoethanol, and 8.4 mM EDTA. |
| T7 | produces 1 nmol of acid-soluble deoxyribonucleotide in |
| Exonuclease | a total reaction volume of 50 μL in 30 minutes at 37° C. |
| | in a buffer of 50 mM potassium acetate, 20 mM Tris- |
| | acetate, 10 mM magnesium acetate, 1 mM DTT, with a |
| | pH of 7.9 at 25° C. with 0.15 mM sonicated duplex |
| | [3H]-DNA. |

### BRIEF DESCRIPTION OF THE DRAWINGS

FIG. 1 shows the time course of linear RNA (˜2.5 kb in size) digested by Xrn-1.

FIG. 2 shows the results of Xrn-1 digestion of RNAs that have undergone the circularization process without ligase.

FIG. 3 shows the results from DNase I and Xrn-1 treatment of RNAs post-circularization.

FIG. 4 shows the results from a scaled up circularized RNA with enzymatic purification method.

### DETAILED DESCRIPTION

The present disclosure provides methods for producing an enriched a population of circular polyribonucleotides. For example, using the compositions and methods described herein, a population of circular polyribonucleotides may be enriched by providing a splint ligation reaction product including circular polyribonucleotides, linear polyribonucleotides, and linear polydeoxyribonucleotides, and reacting the splint ligation product with DNase 1, wherein the DNase I digests at least a portion of the linear polydeoxyribonucleotide to produce a first digested mixture, and reacting the first digested mixture with an exonuclease (e.g., a 5′ exonuclease or a 3′ exonuclease), wherein the exonuclease (e.g., a 5′ exonuclease or a 3′ exonuclease) digests at least a portion of the linear polyribonucleotides to produce a second digested mixture, which includes the enriched population of circular polyribonucleotides. The present disclosure also provides methods of producing an enriched population of circular polyribonucleotides by providing a linear polyribonucleotide having a 5′ and 3′ end and a polydeoxyribonucleotide that hybridizes to the 3′ end of the linear polyribonucleotide and ligating the 5′ end of the linear polyribonucleotide to the 3′ end of the linear polyribonucleotide to produce a splint ligation reaction product including circular polyribonucleotides, linear polyribonucleotides, and linear polydeoxyribonucleotides; contacting the splint ligation reaction product with DNase I, wherein DNase I digests at least a portion of the polydeoxyribonucleotide to produce a first digested mixture; and contacting the first digested mixture with an exonuclease (e.g., a 5′ exonuclease or a 3′ exonuclease), wherein the exonuclease (e.g., a 5′ exonuclease or a 3′ exonuclease) digest as least a portion of the linear polyribonucleotide to produce a second digested mixture, which includes an enriched population of circular polyribonucleotides.

The splint ligation reaction product may be reacted with the DNase I at a concentration, for a time, and at a temperature that is sufficient to allow for at least a portion of the linear polydeoxyribonucleotides to be digested. The first digested mixture may be reacted with the exonuclease (e.g., a 5′ exonuclease or a 3′ exonuclease) at a concentration, for a time, and at a temperature that is sufficient to allow for at least a portion of the linear polyribonucleotides to be digested.

### Methods of Enriching for Circular Polyribonucleotide

In one aspect the disclosure provides a method of producing an enriched population of circular polyribonucleotides including providing a splint ligation reaction product, including circular polyribonucleotides, linear polyribonucleotides, and linear polydeoxyribonucleotides; reacting the splint ligation reaction product with DNase I, wherein DNase I digests at least a portion of the linear polydeoxyribonucleotides to produce a first digested mixture; and reacting the first digested mixture with an exonuclease (e.g., a 5′ exonuclease or a 3′ exonuclease), wherein the exonuclease digests at least a portion of the linear polyribonucleotides to produce a second digested mixture, wherein the second digested mixture includes an enriched population of circular polyribonucleotides.

In another aspect, the disclosure provides a method of producing an enriched population of circular polyribonucleotides including providing a linear polyribonucleotide having a 5′ and 3′ end and a polydeoxyribonucleotide that has a first region that hybridizes to the 5′ end of the linear polyribonucleotide and a second region that hybridizes to the 3′ end of the linear polyribonucleotide; ligating the 5′ end of the linear polyribonucleotide to the 3′ end of the linear polyribonucleotide to produce a splint ligation reaction product including circular polyribonucleotides, linear polyribonucleotides, and linear polydeoxyribonucleotides; reacting the splint ligation reaction product with DNase I, wherein the DNase I digests at least a portion of the polydeoxyribonucleotide to produce a first digested mixture; reacting the first digested mixture with an exonuclease (e.g., a 5′ exonuclease or a 3′ exonuclease), wherein the exonuclease digests at least a portion of the linear polyribonucleotide to produce a second digested mixture, wherein the second digested mixture includes an enriched population of circular polyribonucleotides.

### DNase I Digestion

In an aspect, the splint ligation reaction product, including circular polyribonucleotides, linear polyribonucleotides, and linear polydeoxyribonucleotides, is reacted with DNase I. In some embodiments, the DNase I digests at least a portion of the linear polydeoxyribonucleotides.

In some embodiments, the DNase I is in an amount of between 0.1 U/μg and 1 U/μg (e.g., between 0.1 U/μg and 0.8 U/μg, between 0.1 U/μg and 0.6 U/μg, between 0.1 U/μg and 0.4 U/μg, between 0.1 U/μg and 0.2 U/μg, between 0.2 U/μg and 1 U/μg, between 0.4 U/μg and 1 U/μg, between 0.6 U/μg and 1 U/μg, or between 0.8 U/μg and 1 U/μg). In some embodiments, the DNase I digestion of the splint ligation reaction product is performed for at least 10 minutes (e.g. at least 15 minutes, at least 30 minutes, at least 45 minutes, at least 1 hour, at least 2 hours, between 10 minutes and 24 hours, between 10 minutes and 18 hours, between 10 minutes and 12 hours, between 10 minutes and 6 hours, between 10 minutes and 2 hours, 10 minutes and 1 hour, between 10 minutes and 45 minutes, between 10 minutes and 30 minutes, between 10 minutes and 15 minutes). In some embodiments, the reaction of DNase I with the splint ligation product is performed at a temperature between 30° C. and 42° C. (e.g., about 31° C., about 32° C., about 33° C., about 34° C., about 35° C., about 36° C., about 37° C., about 38° C., about 39° C., about 40° C., and about 41° C.). In some embodiments, the reaction of DNase I with the splint ligation product is performed at a temperature of about 37° C.

### Exonuclease Digestion

In an aspect, the first digested mixture, produced from reacting the splint ligation mixture with DNase I, is reacted with an exonuclease. In some embodiments, the exonuclease digests at least a portion of the linear polyribonucleotides.

In some embodiments, the exonuclease is an exoribonuclease. In some embodiments, the exonuclease is a 5′ exonuclease or a 3′ exonuclease. In some embodiments, the exonuclease is Xrn-1, RNase R, Exonuclease T, A Exonuclease, Exonuclease VII, T7 Exonuclease, Polynucleotide Phosphorylase, RNase D, and Exoribonuclease II. In some embodiments, the exonuclease is a 5′ exonuclease. In some embodiments, the 5′ exonuclease is a 5′-phosphate dependent exonuclease (e.g., Xm-1 or Terminator™ exonuclease). In some embodiments, the 5′ exonuclease is Xrn-1. In some embodiments, the 5′ exonuclease is Terminator™ exonuclease. In some embodiments, the 5′ exonuclease is λ Exonuclease. In some embodiments, the 5′ exonuclease is T7 Exonuclease. In some embodiments, the 5′ exonuclease is Exonuclease VII. In some embodiments, the exonuclease is a 3′ exonuclease. In some embodiments, the 3′ exonuclease is Exonuclease T. In some embodiments, the 3′ exonuclease is Polynucleotide Phosphorylase. In some embodiments, the 3′ exonuclease is RNase D. In some embodiments, the 3′ exonuclease is RNase R. In some embodiments, the 3′ exonuclease is Exoribonuclease II.

In some embodiments, the exonuclease is in amount of between 0.1 U/μg and 1 U/μg (e.g., between 0.1 U/μg and 0.8 U/μg, between 0.1 U/μg and 0.6 U/μg, between 0.1 U/μg and 0.4 U/μg, between 0.1 U/μg and 0.2 U/μg, between 0.2 U/μg and 1 U/μg, between 0.4 U/μg and 1 U/μg, between 0.6 U/μg and 1 U/μg, or between 0.8 U/μg and 1 U/μg). In some embodiments, the Xrn-1 is in amount of between 0.1 U/μg and 1 U/μg (e.g., between 0.1 U/μg and 0.8 U/μg, between 0.1 U/μg and 0.6 U/μg, between 0.1 U/μg and 0.4 U/μg, between 0.1 U/μg and 0.2 U/μg, between 0.2 U/μg and 1 U/μg, between 0.4 U/μg and 1 U/μg, between 0.6 U/μg and 1 U/μg, or between 0.8 U/μg and 1 U/μg). In some embodiments, the reaction of the exonuclease with the first digested mixture, produced by reacting the splint ligation mixture with DNase I, is performed for at least 1 hour (e.g., between 1 hour and 24 hours, between 1 hour and 20 hours, between 1 hour and 16 hours, between 1 hour and 12 hours, between 1 hour and 11 hours, between 1 hour and 10 hours, between 1 hour and 9 hours, between 1 hour and 8 hours, between 1 hour and 7 hours, between 1 hour and 6 hours, between 1 hour and 5 hours, between 1 hour and 4 hours, between 1 hour and 3 hours, and between 1 hour and 2 hours). In some embodiments, the reaction of the exonuclease with the first digested mixture, produced by reacting the splint ligation mixture with DNase I, is performed at a temperature of between 30° C. and 42° C. (e.g., about 31° C., about 32° C., about 33° C., about 34° C., about 35° C., about 36° C., about 37° C., about 38° C., about 39° C., about 40° C., and about 41° C.). In some embodiments, the reaction of the exonuclease with the first digested mixture, produced by reacting the splint ligation mixture with DNase I, is performed at a temperature of about 37° C.

### Circular Polyribonucleotides

The present disclosure provides a population of circular polyribonucleotides that may be enriched from a mixture of circular polyribonucleotides, linear polyribonucleotides, and linear polydeoxyribonucleotides.

In some embodiments, the circular polyribonucleotide includes one or more of the elements as described herein. In some embodiments, the circular polyribonucleotide lacks a poly-A sequence (e.g., lacks a polyA sequence at the 3′ end of the ORF), lacks a free 3′ end, lacks an RNA polymerase recognition motif, or any combination thereof. In some embodiments, the circular polyribonucleotide includes any feature, or any combination of features as disclosed in International Patent Publication No. WO2019/118919, which is hereby incorporated by reference in its entirety.

In some embodiments, the polyribonucleotide (e.g., the circular polyribonucleotide) is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

In some embodiments, the polyribonucleotide (e.g., the circular polyribonucleotide) may be of a sufficient size to accommodate a binding site for a ribosome. In some embodiments, the maximum size of a circular polyribonucleotide can be as large as is within the technical constraints of producing a circular polyribonucleotide, and/or using the circular polyribonucleotide. Without wishing to be bound by any particular theory, it is possible that multiple segments of RNA may be produced from DNA and their 5′ and 3′ free ends annealed to produce a “string” of RNA, which ultimately may be circularized when only one 5′ and one 3′ free end remains. In some embodiments, the maximum size of a circular polyribonucleotide may be limited by the ability of packaging and delivering the RNA to a target. In some embodiments, the size of a circular polyribonucleotide is a length sufficient to encode useful polypeptides, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 400 nucleotides, at least 300 nucleotides, at least 200 nucleotides, at least 100 nucleotides, or at least 70 nucleotides, may be useful.

In some embodiments, the circular polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent, e.g., that is comparable to or similar to in the absence of exonuclease. In some embodiments, the circular polyribonucleotide is not degraded by exonucleases. In some embodiments, the circular polyribonucleotide has reduced degradation when exposed to exonuclease. In some embodiments, the circular polyribonucleotide lacks binding to a cap-binding protein. In some embodiments, the circular polyribonucleotide lacks a 5′ cap.

### Expression Sequences

In some embodiments, the circular polyribonucleotide includes an expression sequence that encodes a peptide or polypeptide. In some embodiments, the circular polyribonucleotide includes at least one expression sequence that encodes a peptide or polypeptide. Such peptide may include, but is not limited to, small peptide, peptidomimetic (e.g., peptoid), amino acids, and amino acid analogs. The peptide may be linear or branched. Such peptide may have a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. Such peptide may include, but is not limited to, a neurotransmitter, a hormone, a drug, a toxin, a viral or microbial particle, a synthetic molecule, and agonists or antagonists thereof.

The encoded polypeptide may have a length from about 5 to about 40,000 amino acids, about 15 to about 35,000 amino acids, about 20 to about 30,000 amino acids, about 25 to about 25,000 amino acids, about 50 to about 20,000 amino acids, about 100 to about 15,000 amino acids, about 200 to about 10,000 amino acids, about 500 to about 5,000 amino acids, about 1,000 to about 2,500 amino acids, or any range therebetween. In some embodiments, the polypeptide has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, or less may be useful.

The polypeptide may be produced in substantial amounts. As such, the polypeptide may be any proteinaceous molecule that can be produced. A polypeptide can be a polypeptide that can be secreted from a cell, or localized to the cytoplasm, nucleus, or membrane compartment of a cell. Some polypeptides include, but are not limited to, at least a portion of a viral envelope protein, metabolic regulatory enzymes (e.g., that regulate lipid or steroid production), an antigen, a cytokine, a toxin, enzymes whose absence is associated with a disease, and polypeptides that are not active in an animal until cleaved (e.g., in the gut of an animal), and a hormone.

In some embodiments, the circular polyribonucleotide includes an expression sequence encoding a protein e.g., a therapeutic protein. In some embodiments, therapeutic proteins that can be expressed from the circular polyribonucleotide disclosed herein have antioxidant activity, binding, cargo receptor activity, catalytic activity, molecular carrier activity, molecular function regulator, molecular transducer activity, nutrient reservoir activity, protein tag, structural molecule activity, toxin activity, transcription regulator activity, translation regulator activity, or transporter activity. Some examples of therapeutic proteins may include, but are not limited to, an enzyme replacement protein, a protein for supplementation, a protein vaccination, antigens (e.g. tumor antigens, viral, bacterial), hormones, cytokines, antibodies, immunotherapy (e.g. cancer), cellular reprogramming/transdifferentiation factor, transcription factors, chimeric antigen receptor, transposase or nuclease, immune effector (e.g., influences susceptibility to an immune response/signal), a regulated death effector protein (e.g., an inducer of apoptosis or necrosis), a non-lytic inhibitor of a tumor (e.g., an inhibitor of an oncoprotein), an epigenetic modifying agent, epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand or a receptor, and a CRISPR system or component thereof.

In some embodiments, exemplary proteins that can be expressed from the circular polyribonucleotide disclosed herein include human proteins, for instance, receptor binding protein, hormone, growth factor, growth factor receptor modulator, and regenerative protein (e.g., proteins implicated in proliferation and differentiation, e.g., therapeutic protein, for wound healing). In some embodiments, exemplary proteins that can be expressed from the circular polyribonucleotide disclosed herein include EGF (epithelial growth factor). In some embodiments, exemplary proteins that can be expressed from the circular polyribonucleotide disclosed herein include enzymes, for instance, oxidoreductase enzymes, metabolic enzymes, mitochondrial enzymes, oxygenases, dehydrogenases, ATP-independent enzyme, and desaturases. In some embodiments, exemplary proteins that can be expressed from the circular polyribonucleotide disclosed herein include an intracellular protein or cytosolic protein. In some embodiments, the circular polyribonucleotide expresses a NanoLuc luciferase (nLuc). In some embodiments, exemplary proteins that can be expressed from the circular polyribonucleotide disclosed herein include a secretary protein, for instance, a secretary enzyme. In some cases, the circular polyribonucleotide expresses a secretary protein that can have a short half-life therapeutic in the blood, or can be a protein with a subcellular localization signal, or protein with secretory signal peptide. In some embodiments, the circular polyribonucleotide expresses a Gaussia Luciferase (gLuc). In some cases, the circular polyribonucleotide expresses a non-human protein, for instance, a fluorescent protein, an energy-transfer acceptor, or a protein-tag like Flag, Myc, or His. In some embodiments, exemplary proteins that can be expressed from the circular polyribonucleotide include a GFP. In some embodiments, the circular polyribonucleotide expresses tagged proteins, e.g., fusion proteins or engineered proteins containing a protein tag, e.g., chitin binding protein (CBP), maltose binding protein (MBP), Fc tag, glutathione-S-transferase (GST), AviTag, Calmodulin-tag, polyglutamate tag; E-tag, FLAG-tag), HA-tag, His-tag, Myc-tag, NE-tag, S-tag, SBP-tag, Softag 1, Softag 3, Spot-tag, Strep-tag; TC tag, Ty tag, V5 tag; VSV-tag; or Xpress tag.

In some embodiments, the circular polyribonucleotide encodes the expression of an antibody, e.g., an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can comprise more than one expression sequence, each of which expresses a portion of an antibody, and the sum of which can constitute the antibody. In some cases, the circular polyribonucleotide comprises one expression sequence coding for the heavy chain of an antibody, and another expression sequence coding for the light chain of the antibody. In some cases, when the circular polyribonucleotide is expressed in a cell or a cell-free environment, the light chain and heavy chain can be subject to appropriate modification, folding, or other post-translation modification to form a functional antibody.

### Regulatory Elements

In some embodiments, a circular polyribonucleotide includes a regulatory element, e.g., a sequence that modifies expression of an expression sequence within the circular polyribonucleotide. A regulatory element may include a sequence that is located adjacent to an expression sequence that encodes an expression product. A regulatory element may be operably linked to the adjacent sequence. A regulatory element may increase an amount of product expressed as compared to an amount of the expressed product when no regulatory element is present. A regulatory element may be used to increase the expression of one or more polypeptides encoded by a circular polyribonucleotide. Likewise, a regulatory element may be used to decrease the expression of one or more polypeptides encoded by a circular polyribonucleotide. In some embodiments, a regulatory element may be used to increase expression of a polypeptide and another regulatory element may be used to decrease expression of another polypeptide on the same circular polyribonucleotide. In addition, one regulatory element can increase an amount of a product (e.g., a polypeptide) expressed for multiple expression sequences attached in tandem. Hence, one regulatory element can enhance the expression of one or more expression sequences (e.g., polypeptides). Multiple regulatory elements can also be used, for example, to differentially regulate expression of different expression sequences. In some embodiments, a regulatory element as provided herein can include a selective translation sequence. As used herein, the term “selective translation sequence” refers to a nucleic acid sequence that selectively initiates or activates translation of an expression sequence in the circular polyribonucleotide, for instance, certain riboswitch aptazymes. A regulatory element can also include a selective degradation sequence. As used herein, the term “selective degradation sequence” refers to a nucleic acid sequence that initiates degradation of the circular polyribonucleotide, or an expression product of the circular polyribonucleotide. In some embodiments, the regulatory element is a translation modulator. A translation modulator can modulate translation of the expression sequence in the circular polyribonucleotide. A translation modulator can be a translation enhancer or suppressor. In some embodiments, a translation initiation sequence can function as a regulatory element. Further examples of regulatory elements are described in paragraphs [0154]-[0161] of International Patent Publication No. WO2019/118919, which is hereby incorporated by reference in its entirety.

Nucleotides flanking a codon that initiates translation, such as, but not limited to, a start codon or an alternative start codon, are known to affect the translation efficiency, the length and/or the structure of the circular polyribonucleotide. (See e.g., Matsuda and Mauro PLoS ONE, 2010 5: 11; the contents of which are herein incorporated by reference in its entirety). Masking any of the nucleotides flanking a codon that initiates translation may be used to alter the position of translation initiation, translation efficiency, length and/or structure of the circular polyribonucleotide.

In one embodiment, a masking agent may be used near the start codon or alternative start codon in order to mask or hide the codon to reduce the probability of translation initiation at the masked start codon or alternative start codon. In another embodiment, a masking agent may be used to mask a start codon of the circular polyribonucleotide in order to increase the likelihood that translation will initiate at an alternative start codon.

### Translation Initiation Sequences

In some embodiments, a circular polyribonucleotide encodes a polypeptide and includes a translation initiation sequence, e.g., a start codon. In some embodiments, the translation initiation sequence includes a Kozak or Shine-Dalgarno sequence. In some embodiments, the translation initiation sequence includes a Kozak sequence. In some embodiments, the circular polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the circular polyribonucleotide includes at least one translation initiation sequence adjacent to an expression sequence. In some embodiments, the translation initiation sequence provides conformational flexibility to the circular polyribonucleotide. In some embodiments, the translation initiation sequence is within a substantially single stranded region of the circular polyribonucleotide. Further examples of translation initiation sequences are described in paragraphs [0163]-[0165] of International Patent Publication No. WO2019/118919, which is hereby incorporated by reference in its entirety.

The circular polyribonucleotide may include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. Translation may initiate on the first start codon or may initiate downstream of the first start codon.

In some embodiments, a circular polyribonucleotide may initiate at a codon which is not the first start codon, e.g., AUG. Translation of the circular polyribonucleotide may initiate at an alternative translation initiation sequence, such as those described in [0164] of International Patent Publication No. WO2019/118919, which is incorporated herein by reference in its entirety.

In some embodiments, translation is initiated by eukaryotic initiation factor 4A (eIF4A) treatment with Rocaglates (translation is repressed by blocking 43S scanning, leading to premature, upstream translation initiation and reduced protein expression from transcripts bearing the RocA-eIF4A target sequence, see for example, www.nature.com/articles/nature17978).

### Stagger Elements

A circular polyribonucleotide of the disclosure can include a cleavage domain (e.g., a stagger element or a cleavage sequence). The term “stagger element” refers to a moiety, such as a nucleotide sequence, that induces ribosomal pausing during translation. In some embodiments, the stagger element is a non-conserved sequence of amino-acids with a strong alpha-helical propensity followed by the consensus sequence −D(V/I)ExNPGP (SEQ ID NO: 2), where x=any amino acid. In some embodiments, the stagger element may include a chemical moiety, such as glycerol, a non-nucleic acid linking moiety, a chemical modification, a modified nucleic acid, or any combination thereof.

In some embodiments, a circular polyribonucleotide includes at least one stagger element adjacent to an expression sequence. In some embodiments, the stagger element is present on one or both sides of each expression sequence, leading to separation of the expression products. In some embodiments, the stagger element is a portion of the one or more expression sequences. In some embodiments, the circular polyribonucleotide includes one or more expression sequences, and each of the one or more expression sequences is separated from a succeeding expression sequence. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element includes a portion of an expression sequence of the one or more expression sequences.

Examples of stagger elements are described in paragraphs [0172]-[0175] of International Patent Publication No. WO2019/118919, which is hereby incorporated by reference in its entirety.

To avoid production of a continuous expression product while maintaining rolling circle translation, a stagger element may be included to induce ribosomal pausing during translation. In some embodiments, the stagger element is at the 3′ end of at least one of the one or more expression sequences. The stagger element can be configured to stall a ribosome during rolling circle translation of the circular polyribonucleotide. The stagger element may include, but is not limited to a 2A-like, or CHYSEL (cis-acting hydrolase element) sequence. In some embodiments, the stagger element encodes a sequence with a C-terminal consensus sequence that is X₁X₂X₃EX₅NPGP (SEQ ID NO: 22), where X₁ is absent or G or H, X₂ is absent or D or G, X₃ is D or V or I or S or M, and X₅ is any amino acid. In some embodiments, this sequence includes a non-conserved sequence of amino-acids with a strong alpha-helical propensity followed by the consensus sequence −D(V/I)ExNPGP (SEQ ID NO: 2), where x=any amino acid. Some non-limiting examples of stagger elements includes GDVESNPGP (SEQ ID NO: 3), GDIEENPGP (SEQ ID NO: 4), VEPNPGP (SEQ ID NO: 5), IETNPGP (SEQ ID NO: 6), GDIESNPGP (SEQ ID NO: 7), GDVELNPGP (SEQ ID NO: 8), GDIETNPGP (SEQ ID NO: 9), GDVENPGP (SEQ ID NO: 10), GDVEENPGP (SEQ ID NO: 11), GDVEQNPGP (SEQ ID NO: 12), IESNPGP (SEQ ID NO: 13), GDIELNPGP (SEQ ID NO: 14), HDIETNPGP (SEQ ID NO: 15), HDVETNPGP (SEQ ID NO: 16), HDVEMNPGP (SEQ ID NO: 17), GDMESNPGP (SEQ ID NO: 18), GDVETNPGP (SEQ ID NO: 19), GDIEQNPGP ((SEQ ID NO: 20), and DSEFNPGP (SEQ ID NO: 21).

In some embodiments, a stagger element described herein cleaves an expression product, such as between G and P of the consensus sequence described herein. As one non-limiting example, the circular polyribonucleotide includes at least one stagger element to cleave the expression product. In some embodiments, the circular polyribonucleotide includes a stagger element adjacent to at least one expression sequence. In some embodiments, the circular polyribonucleotide includes a stagger element after each expression sequence. In some embodiments, the circular polyribonucleotide includes a stagger element which is present on one or both sides of each expression sequence, leading to translation of individual peptide(s) and or polypeptide(s) from each expression sequence.

In some embodiments, a stagger element includes one or more modified nucleotides or unnatural nucleotides that induce ribosomal pausing during translation. Unnatural nucleotides may include peptide nucleic acid (PNA), Morpholino and locked nucleic acid (LNA), as well as glycol nucleic acid (GNA) and threose nucleic acid (TNA). Examples such as these are distinguished from naturally occurring DNA or RNA by changes to the backbone of the molecule. Modifications can include any modification to the sugar, the nucleobase, the internucleoside linkage (e.g., to a linking phosphate/to a phosphodiester linkage/to the phosphodiester backbone), and any combination thereof that can induce ribosomal pausing during translation. Some of the exemplary modifications provided herein are described elsewhere herein.

In some embodiments, a stagger element is present in a circular polyribonucleotide in other forms. For example, in some exemplary circular polyribonucleotides, a stagger element includes a termination element of a first expression sequence in the circular polyribonucleotide, and a nucleotide spacer sequence that separates the termination element from a first translation initiation sequence of an expression succeeding the first expression sequence. In some examples, the first stagger element of the first expression sequence is upstream of (5′ to) a first translation initiation sequence of the expression succeeding the first expression sequence in the circular polyribonucleotide. In some cases, the first expression sequence and the expression sequence succeeding the first expression sequence are two separate expression sequences in the circular polyribonucleotide. The distance between the first stagger element and the first translation initiation sequence can enable continuous translation of the first expression sequence and its succeeding expression sequence. In some embodiments, the first stagger element includes a termination element and separates an expression product of the first expression sequence from an expression product of its succeeding expression sequences, thereby creating discrete expression products. In some cases, the circular polyribonucleotide including the first stagger element upstream of the first translation initiation sequence of the succeeding sequence in the circular polyribonucleotide is continuously translated, while a corresponding circular polyribonucleotide including a stagger element of a second expression sequence that is upstream of a second translation initiation sequence of an expression sequence succeeding the second expression sequence is not continuously translated. In some cases, there is only one expression sequence in the circular polyribonucleotide, and the first expression sequence and its succeeding expression sequence are the same expression sequence. In some exemplary circular polyribonucleotides, a stagger element includes a first termination element of a first expression sequence in the circular polyribonucleotide, and a nucleotide spacer sequence that separates the termination element from a downstream translation initiation sequence. In some such examples, the first stagger element is upstream of (5′ to) a first translation initiation sequence of the first expression sequence in the circular polyribonucleotide. In some cases, the distance between the first stagger element and the first translation initiation sequence enables continuous translation of the first expression sequence and any succeeding expression sequences. In some embodiments, the first stagger element separates one round expression product of the first expression sequence from the next round expression product of the first expression sequences, thereby creating discrete expression products. In some cases, the circular polyribonucleotide including the first stagger element upstream of the first translation initiation sequence of the first expression sequence in the circular polyribonucleotide is continuously translated, while a corresponding circular polyribonucleotide including a stagger element upstream of a second translation initiation sequence of a second expression sequence in the corresponding circular polyribonucleotide is not continuously translated. In some cases, the distance between the second stagger element and the second translation initiation sequence is at least 2×, 3×, 4×, 5×, 6×, 7×, 8×, 9×, or 10× greater in the corresponding circular polyribonucleotide than a distance between the first stagger element and the first translation initiation in the circular polyribonucleotide. In some cases, the distance between the first stagger element and the first translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater. In some embodiments, the distance between the second stagger element and the second translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, nt, 35 nt, 40 nt, 45 nt, 50 nt, 55 nt, 60 nt, 65 nt, 70 nt, 75 nt, or greater than the distance between the first stagger element and the first translation initiation. In some embodiments, the circular polyribonucleotide includes more than one expression sequence.

### IRES

In some embodiments, a circular polyribonucleotide described herein includes an internal ribosome entry site (IRES) element. In some embodiments, a circular polyribonucleotide described herein includes more than one (e.g., 2, 3, 4, and 5) internal ribosome entry site (IRES) element. In some embodiments, the circular polyribonucleotide includes one or more IRES sequences on one or both sides of each expression sequence, leading to separation of the resulting peptide(s) and or polypeptide(s). In some embodiments, the IRES flanks both sides of at least one (e.g., 2, 3, 4, 5 or more) expression sequence. A suitable IRES element to include in a circular polyribonucleotide can be an RNA sequence capable of engaging a eukaryotic ribosome. In some embodiments, the IRES is an encephalomyocarditis virus (EMCV) IRES. In some embodiments, the IRES is a Coxsackievirus (CVB3) IRES. Further examples of an IRES are described in paragraphs [0166]-[0168] of International Patent Publication No. WO2019/118919, which is hereby incorporated by reference in its entirety. In some embodiments, the circular polyribonucleotide lacks an internal ribosomal entry site. In some embodiments, the circular polyribonucleotide lacks an internal ribosomal entry site and is competent for protein expression from its one or more expression sequences.

### Translation

In some embodiments, once translation of a circular polyribonucleotide is initiated, the ribosome bound to the circular polyribonucleotide does not disengage from the circular polyribonucleotide before finishing at least one round of translation of the circular polyribonucleotide. In some embodiments, the circular polyribonucleotide as described herein is competent for rolling circle translation. In some embodiments, during rolling circle translation, once translation of the circular polyribonucleotide is initiated, the ribosome bound to the circular polyribonucleotide does not disengage from the circular polyribonucleotide before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1000 rounds, at least 1500 rounds, at least 2000 rounds, at least 5000 rounds, at least 10000 rounds, at least 105 rounds, or at least 106 rounds of translation of the circular polyribonucleotide.

In some embodiments, the rolling circle translation of a circular polyribonucleotide leads to generation of polypeptide product that is translated from more than one round of translation of the circular polyribonucleotide (“continuous” expression product). In some embodiments, the circular polyribonucleotide includes a stagger element, and rolling circle translation of the circular polyribonucleotide leads to generation of polypeptide product that is generated from a single round of translation or less than a single round of translation of the circular polyribonucleotide (“discrete” expression product). In some embodiments, the circular polyribonucleotide is configured such that at least 10%, 20%, 30%, 40%, 50%, at least 60%, at least 70%, at least 80%, at least 90%, at least 95%, at least 96%, at least 97%, at least 98%, at least 99%, or 100% of total polypeptides (molar/molar) generated during the rolling circle translation of the circular polyribonucleotide are discrete polypeptides. In some embodiments, the circular polyribonucleotide is configured such that at least 99% of the total polypeptides are discrete polypeptides. In some embodiments, the amount ratio of the discrete products over the total polypeptides is tested in an in vitro translation system. In some embodiments, the in vitro translation system used for the test of amount ratio includes rabbit reticulocyte lysate. In some embodiments, the amount ratio is tested in an in vivo translation system, such as a eukaryotic cell or a prokaryotic cell, a cultured cell, or a cell in an organism.

### Untranslated Regions

In some embodiments, a circular polyribonucleotide includes untranslated regions (UTRs). UTRs of a genomic region including a gene may be transcribed but not translated. In some embodiments, a UTR may be included upstream of the translation initiation sequence of an expression sequence described herein. In some embodiments, a UTR may be included downstream of an expression sequence described herein. In some instances, one UTR for a first expression sequence is the same as or continuous with or overlapping with another UTR for a second expression sequence. In some embodiments, the intron is a human intron. In some embodiments, the intron is a full-length human intron, e.g., ZKSCAN1.

In some embodiments, a circular polyribonucleotide includes a UTR with one or more stretches of adenosines and uridines embedded within. These AU rich signatures may increase turnover rates of the expression product.

Introduction, removal, or modification of UTR AU rich elements (AREs) may be useful to modulate the stability, or immunogenicity (e.g., the level of one or more marker of an immune or inflammatory response) of the circular polyribonucleotide. When engineering specific circular polyribonucleotides, one or more copies of an ARE may be introduced to the circular polyribonucleotide and the copies of an ARE may modulate translation and/or production of an expression product. Likewise, AREs may be identified and removed or engineered into the circular polyribonucleotide to modulate the intracellular stability and thus affect translation and production of the resultant protein.

It should be understood that any UTR from any gene may be incorporated into the respective flanking regions of the circular polyribonucleotide. Exemplary untranslated regions are described in paragraphs [0197]-[201] of International Patent Publication No. WO2019/118919, which is hereby incorporated by reference in its entirety.

In some embodiments, a circular polyribonucleotide lacks a 5′-UTR and is competent for protein expression from its one or more expression sequences. In some embodiments, the circular polyribonucleotide lacks a 3′-UTR and is competent for protein expression from its one or more expression sequences. In some embodiments, a circular polyribonucleotide lacks a 5′-UTR. In some embodiments, the circular polyribonucleotide lacks a 3′-UTR. In some embodiments, the circular polyribonucleotide lacks a termination element and is competent for protein expression from its one or more expression sequences. In some embodiments, the circular polyribonucleotide lacks a cap and is competent for protein expression from its one or more expression sequences. In some embodiments, the circular polyribonucleotide lacks a 5′-UTR, a 3′-UTR, and an IRES, and is competent for protein expression from its one or more expression sequences. In some embodiments, the circular polyribonucleotide includes one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (e.g., siRNA, lncRNAs, shRNA), and a sequence that encodes a therapeutic mRNA or protein.

### Termination Elements

A circular polyribonucleotide can include one or more expression sequences and each expression sequence may or may not have a termination element. Further examples of termination elements are described in paragraphs [0169]-[0170] of International Patent Publication No. WO2019/118919, which is hereby incorporated by reference in its entirety.

In some embodiments, a circular polyribonucleotide includes a poly-A sequence. In some embodiments, the length of a poly-A sequence is greater than 10 nucleotides in length. In one embodiment, the poly-A sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A sequence is designed according to the descriptions of the poly-A sequence in [0202]-[0204] of International Patent Publication No. WO2019/118919, which is incorporated herein by reference in its entirety. In some embodiments, a circular polyribonucleotide lacks a poly-A sequence (e.g., lacks a poly-A sequence at the 3′ end of the ORF). In some embodiments, the circular polyribonucleotide lacks a termination element. In some embodiments, the circular polyribonucleotide lacks a poly-A sequence (e.g., lacks a polyA sequence at the 3′ end of the ORF) and is competent for protein expression from its one or more expression sequences.

In some embodiments, the circular polyribonucleotide includes one or more expression sequences, and the expression sequences lack a termination element, such that the circular polyribonucleotide is continuously translated. Exclusion of a termination element may result in rolling circle translation or continuous expression of expression product, e.g., peptides or polypeptides, due to lack of ribosome stalling or fall-off. In such an embodiment, rolling circle translation expresses a continuous expression product through each expression sequence. In some other embodiments, a termination element of an expression sequence can be part of a stagger element. In some embodiments, one or more expression sequences in the circular polyribonucleotide comprises a termination element. However, rolling circle translation or expression of a succeeding (e.g., second, third, fourth, fifth, etc.) expression sequence in the circular polyribonucleotide is performed. In such instances, the expression product may fall off the ribosome when the ribosome encounters the termination element, e.g., a stop codon, and terminates translation. In some embodiments, translation is terminated while the ribosome, e.g., at least one subunit of the ribosome, remains in contact with the circular polyribonucleotide.

In some embodiments, the circular polyribonucleotide includes a termination element at the end of one or more expression sequences. In some embodiments, one or more expression sequences comprises two or more termination elements in succession. In such embodiments, translation is terminated and rolling circle translation is terminated. In some embodiments, the ribosome completely disengages with the circular polyribonucleotide. In some such embodiments, production of a succeeding (e.g., second, third, fourth, fifth, etc.) expression sequence in the circular polyribonucleotide may require the ribosome to reengage with the circular polyribonucleotide prior to initiation of translation. Generally, termination elements include an in-frame nucleotide triplet that signals termination of translation, e.g., UAA, UGA, UAG. In some embodiments, one or more termination elements in the circular polyribonucleotide are frame-shifted termination elements, such as but not limited to, off-frame or −1 and +1 shifted reading frames (e.g., hidden stop) that may terminate translation. Frame-shifted termination elements include nucleotide triples, TAA, TAG, and TGA that appear in the second and third reading frames of an expression sequence. Frame-shifted termination elements may be important in preventing misreads of mRNA, which is often detrimental to the cell.

### RNA Binding Sites

In some embodiments, the circular polyribonucleotide comprises one or more target RNA binding sites. In some embodiments, the circular polyribonucleotide includes target RNA binding sites that modify expression of an endogenous gene and/or an exogenous gene. In some embodiments, the target RNA binding site modulates expression of a host gene. The target RNA binding site can include a sequence that hybridizes to an endogenous gene (e.g., a sequence for a miRNA, siRNA, mRNA, lncRNA, RNA, DNA, an antisense RNA, gRNA as described herein), a sequence that hybridizes to an exogenous nucleic acid such as a viral DNA or RNA, a sequence that hybridizes to an RNA, a sequence that interferes with gene transcription, a sequence that interferes with RNA translation, a sequence that stabilizes RNA or destabilizes RNA such as through targeting for degradation, or a sequence that modulates a DNA- or RNA-binding factor. In some embodiments, the circular polyribonucleotide comprises a target aptamer sequence that binds to an RNA. The target aptamer sequence can bind to an endogenous gene (e.g., a sequence for a miRNA, siRNA, mRNA, lncRNA, RNA, DNA, an antisense RNA, gRNA as described herein), to an exogenous nucleic acid such as a viral DNA or RNA, to an RNA, to a sequence that interferes with gene transcription, to a sequence that interferes with RNA translation, to a sequence that stabilizes RNA or destabilizes RNA such as through targeting for degradation, or to a sequence that modulates a DNA- or RNA-binding factor. The secondary structure of the target aptamer sequence can bind to the RNA. The circular RNA can form a complex with the RNA by binding of the target aptamer sequence to the RNA.

In some embodiments, the target RNA binding site can be one of a tRNA, lncRNA, lincRNA, miRNA, rRNA, snRNA, microRNA, siRNA, piRNA, snoRNA, snRNA, exRNA, scaRNA, Y RNA, and hnRNA binding site. Target RNA binding sites are well-known to persons of ordinary skill in the art.

Certain target RNA binding sites can inhibit gene expression through the biological process of RNA interference (RNAi). In some embodiments, the circular polyribonucleotides comprises an RNAi molecule with RNA or RNA-like structures typically having 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNA (siRNA), double-strand RNA (dsRNA), microRNA (miRNA), short hairpin RNA (shRNA), meroduplexes, and dicer substrates. Further examples of target binding sites are described in paragraphs [0129]-[0146] of WO2020/023655, which is hereby incorporated by reference in its entirety.

### DNA Binding Sites

In some embodiments, the circular polyribonucleotide comprises a target DNA binding site, such as a sequence for a guide RNA (gRNA). In some embodiments, the circular polyribonucleotide comprises a guide RNA or a complement to a gRNA sequence. A gRNA short synthetic RNA composed of a target binding sequence necessary for binding to the incomplete effector moiety and a user-defined ˜20 nucleotide targeting sequence for a genomic target. Guide RNA sequences can have a length of between 17-24 nucleotides (e.g., 19, 20, or 21 nucleotides) and complementary to the targeted nucleic acid sequence. Custom gRNA generators and algorithms can be used in the design of effective guide RNA. Gene editing can be achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNA can be effective in genome editing.

The gRNA can recognize specific DNA sequences (e.g., sequences adjacent to or within a promoter, enhancer, silencer, or repressor of a gene).

In some embodiments, the gRNA is part of a CRISPR system for gene editing. For gene editing, the circular polyribonucleotide can be designed to include one or multiple guide RNA sequences corresponding to a desired target DNA sequence. The gRNA sequences may include at least 10, 11, 12, 13, 14, 15, 16, 17, 18, 19, 20, 21, 22, 23, 24, 25, 26, 27, 28, 29, 30 or more nucleotides for interaction with Cas9 or other exonuclease to cleave DNA, e.g., Cpf1 interacts with at least about 16 nucleotides of gRNA sequence for detectable DNA cleavage.

In some embodiments, the circular polyribonucleotide comprises a target aptamer sequence that can bind to DNA. The secondary structure of the target aptamer sequence can bind to DNA. In some embodiments, the circular polyribonucleotide forms a complex with the DNA by binding of the target aptamer sequence to the DNA.

Further examples of circular polyribonucleotide sequences that bind to DNA are described in paragraphs [0151]-[0153] of WO2020/023655, which is hereby incorporated by reference in its entirety.

### Protein-Binding

In some embodiments, the circular polyribonucleotide includes one or more protein binding sites that enable a protein, e.g., a ribosome, to bind to an internal site in the RNA sequence. By engineering protein binding sites, e.g., ribosome binding sites, into the circular polyribonucleotide, the circular polyribonucleotide may evade or have reduced detection by the host's immune system, have modulated degradation, or modulated translation, by masking the circular polyribonucleotide from components of the host's immune system.

In some embodiments, the circular polyribonucleotide comprises at least one immunoprotein binding site, for example to evade immune responses, e.g., CTL (cytotoxic T lymphocyte) responses. In some embodiments, the immunoprotein binding site is a nucleotide sequence that binds to an immunoprotein and aids in masking the circular polyribonucleotide as exogenous. In some embodiments, the immunoprotein binding site is a nucleotide sequence that binds to an immunoprotein and aids in hiding the circular polyribonucleotide as exogenous or foreign.

Traditional mechanisms of ribosome engagement to linear RNA involve ribosome binding to the capped 5′ end of an RNA. From the 5′ end, the ribosome migrates to an initiation codon, whereupon the first peptide bond is formed. According to the present invention, internal initiation (i.e., cap-independent) of translation of the circular polyribonucleotide does not require a free end or a capped end. Rather, a ribosome binds to a non-capped internal site, whereby the ribosome begins polypeptide elongation at an initiation codon. In some embodiments, the circular polyribonucleotide includes one or more RNA sequences comprising a ribosome binding site, e.g., an initiation codon.

Natural 5′UTRs bear features which play roles in for translation initiation. They harbor signatures like Kozak sequences which are commonly known to be involved in the process by which the ribosome initiates translation of many genes. Kozak sequences have the consensus CCR(A/G)CCAUGG (SEQ ID NO: 23), where R is a purine (adenine or guanine) three bases upstream of the start codon (AUG), which is followed by another ‘G’. 5′UTR also have been known to form secondary structures which are involved in elongation factor binding.

In some embodiments, the circular polyribonucleotide encodes a protein binding sequence that binds to a protein. In some embodiments, the protein binding sequence targets or localizes the circular polyribonucleotide to a specific target. In some embodiments, the protein binding sequence specifically binds an arginine-rich region of a protein.

In some embodiments, the protein binding site includes, but is not limited to, a binding site to the protein such as ACIN1, AGO, APOBEC3F, APOBEC3G, ATXN2, AUH, BCCIP, CAPRIN1, CELF2, CPSF1, CPSF2, CPSF6, CPSF7, CSTF2, CSTF2T, CTCF, DDX21, DDX3, DDX3X, DDX42, DGCR8, EIF3A, EIF4A3, EIF4G2, ELAVL1, ELAVL3, FAM120A, FBL, FIP1L1, FKBP4, FMR1, FUS, FXR1, FXR2, GNL3, GTF2F1, HNRNPA1, HNRNPA2B1, HNRNPC, HNRNPK, HNRNPL, HNRNPM, HNRNPU, HNRNPUL1, IGF2BP1, IGF2BP2, IGF2BP3, ILF3, KHDRBS1, LARP7, LIN28A, LIN28B, m6A, MBNL2, METTL3, MOV10, MSI1, MSI2, NONO, NONO-, NOP58, NPM1, NUDT21, PCBP2, POLR2A, PRPF8, PTBP1, RBFOX2, RBM10, RBM22, RBM27, RBM47, RNPS1, SAFB2, SBDS, SF3A3, SF3B4, SIRT7, SLBP, SLTM, SMNDC1, SND1, SRRM4, SRSF1, SRSF3, SRSF7, SRSF9, TAF15, TARDBP, TIA1, TNRC6A, TOP3B, TRA2A, TRA2B, U2AF1, U2AF2, UNK, UPF1, WDR33, XRN2, YBX1, YTHDC1, YTHDF1, YTHDF2, YWHAG, ZC3H7B, PDK1, AKT1, and any other protein that binds RNA.

### Modifications

A circular polyribonucleotide described herein may include one or more substitutions, insertions and/or additions, deletions, and covalent modifications with respect to reference sequences, in particular, the parent polyribonucleotide, are included within the scope of this invention.

In some embodiments, a circular polyribonucleotide includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27:196-197). In some embodiments, the first isolated nucleic acid includes messenger RNA (mRNA). In some embodiments, the mRNA includes at least one nucleoside selected from the group such as those described in [0311] of International Patent Publication No. WO2019/118919, which is incorporated herein by reference in its entirety.

A circular polyribonucleotide may include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, a circular polyribonucleotide includes at least one N(6)methyladenosine (m6A) modification to increase translation efficiency. In some embodiments, the N(6)methyladenosine (m6A) modification can reduce immunogenicity (e.g., reduce the level of one or more marker of an immune or inflammatory response) of the circular polyribonucleotide.

In some embodiments, a modification may include a chemical or cellular induced modification. For example, some non-limiting examples of intracellular RNA modifications are described by Lewis and Pan in “RNA modifications and structures cooperate to guide RNA-protein interactions” from Nat Reviews Mol Cell Biol, 2017, 18:202-210.

In some embodiments, chemical modifications to the ribonucleotides of a circular polyribonucleotide or oligonucleotides may enhance immune evasion. The circular polyribonucleotide or may be synthesized and/or modified by methods well established in the art, such as those described in “Current protocols in nucleic acid chemistry,” Beaucage, S. L. et al. (Eds.), John Wiley & Sons, Inc., New York, NY, USA, which is hereby incorporated herein by reference. Modifications include, for example, end modifications, e.g., 5′ end modifications (phosphorylation (mono-, di- and tri-), conjugation, inverted linkages, etc.), 3 end modifications (conjugation, DNA nucleotides, inverted linkages, etc.), base modifications (e.g., replacement with stabilizing bases, destabilizing bases, or bases that base pair with an expanded repertoire of partners), removal of bases (abasic nucleotides), or conjugated bases. The modified ribonucleotide bases may also include 5-methylcytidine and pseudouridine. In some embodiments, base modifications may modulate expression, immune response, stability, subcellular localization, to name a few functional effects, of the circular polyribonucleotide. In some embodiments, the modification includes a bi-orthogonal nucleotide, e.g., an unnatural base. See for example, Kimoto et al, Chem Commun (Camb), 2017, 53:12309, DOI: 10.1039/c7cc06661a, which is hereby incorporated by reference.

In some embodiments, sugar modifications (e.g., at the 2′ position or 4′ position) or replacement of the sugar one or more ribonucleotides of the circular polyribonucleotide or oligonucleotide may, as well as backbone modifications, include modification or replacement of the phosphodiester linkages. Specific examples of circular polyribonucleotide include, but are not limited to, circular polyribonucleotide including modified backbones or no natural internucleoside linkages such as internucleoside modifications, including modification or replacement of the phosphodiester linkages. Circular polyribonucleotides having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, the circular polyribonucleotide will include ribonucleotides with a phosphorus atom in its internucleoside backbone.

Modified circular polyribonucleotide or oligonucleotide backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the circular polyribonucleotide may be negatively or positively charged.

The modified nucleotides, which may be incorporated into the circular polyribonucleotide or oligonucleotide, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The a-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

Phosphorothioate linked to the circular polyribonucleotide is expected to reduce the innate immune response through weaker binding/activation of cellular innate immune molecules.

In specific embodiments, a modified nucleoside includes an alpha-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, a circular polyribonucleotide or oligonucleotide may include one or more cytotoxic nucleosides. For example, cytotoxic nucleosides may be incorporated into circular polyribonucleotide, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(IH,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

A circular polyribonucleotide or oligonucleotide may or may not be uniformly modified along the entire length of the molecule. For example, one or more or all types of nucleotides (e.g., naturally-occurring nucleotides, purine or pyrimidine, or any one or more or all of A, G, U, C, I, pU) may or may not be uniformly modified in the circular polyribonucleotide or oligonucleotides, or in a given predetermined sequence region thereof. In some embodiments, the circular polyribonucleotide or oligonucleotide includes a pseudouridine. In some embodiments, the circular polyribonucleotide or oligonucleotide includes an inosine, which may aid in the immune system characterizing the circular polyribonucleotide as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

In some embodiments, all nucleotides in a circular polyribonucleotide or oligonucleotide (or in a given sequence region thereof) are modified. In some embodiments, the modification may include an m6A, which may augment expression; an inosine, which may attenuate an immune response; pseudouridine, which may increase RNA stability, or translational readthrough (stagger element), an m5C, which may increase stability; and a 2,2,7-trimethylguanosine, which aids subcellular translocation (e.g., nuclear localization).

Different sugar modifications, nucleotide modifications, and/or internucleoside linkages (e.g., backbone structures) may exist at various positions in a circular polyribonucleotide or oligonucleotide. One of ordinary skill in the art will appreciate that the nucleotide analogs or other modification(s) may be located at any position(s) of the circular polyribonucleotide or oligonucleotide, such that the function of the circular polyribonucleotide is not substantially decreased. A modification may also be a non-coding region modification. The circular polyribonucleotide or oligonucleotide may include from about 1% to about 100% modified nucleotides (either in relation to overall nucleotide content, or in relation to one or more types of nucleotide, i.e. any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

### Production Methods

In some embodiments, a circular polyribonucleotide includes a deoxyribonucleic acid sequence that is non-naturally occurring and can be produced using recombinant technology (e.g., derived in vitro using a DNA plasmid), chemical synthesis, or a combination thereof.

It is within the scope of the disclosure that a DNA molecule used to produce an RNA circle can include a DNA sequence of a naturally-occurring original nucleic acid sequence, a modified version thereof, or a DNA sequence encoding a synthetic polypeptide not normally found in nature (e.g., chimeric molecules or fusion proteins). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof.

In some embodiments, a linear primary construct or linear mRNA may be cyclized, or concatemerized to create a circular polyribonucleotide described herein. The mechanism of cyclization or concatemerization may occur through methods such as splint ligation methods. The newly formed 5′-/3′-linkage may be an intramolecular linkage or an intermolecular linkage.

Methods of making circular polyribonucleotides described herein are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

### Circularization

In an aspect, the disclosure provides a method of producing an enriched population of circular polyribonucleotides. In some embodiments, a linear polyribonucleotide for circularization may be cyclized, or concatemerized. In some embodiments, the linear polyribonucleotide for circularization may be cyclized in vitro prior to formulation and/or delivery. In some embodiments, the linear polyribonucleotide for circularization may be cyclized within a cell.

### Extracellular Circularization

In some embodiments, the disclosure provides a method of producing an enriched population of circular polyribonucleotides. In some embodiments, the circular polyribonucleotides are produced by providing a linear polyribonucleotide having a 5′ and 3′ end and a polydeoxyribonucleotide that has a first region that hybridizes to the 5′ end of the linear polyribonucleotide and a second region that hybridizes to the 3′ end of the linear polyribonucleotide. In some embodiments, the 5′ end of the linear polyribonucleotide is then ligated to the 3′ end of the linear polyribonucleotide to produce a splint ligation reaction product including circular polyribonucleotides, linear polyribonucleotides, and linear polydeoxyribonucleotides.

In some embodiments, the ligation is splint ligation. For example, a splint ligase, like SplintR® ligase, RNA ligase II, T4 RNA ligase, or T4 DNA ligase, can be used for splint ligation. For splint ligation, a single stranded polynucleotide (splint), like a single stranded RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circular polyribonucleotide.

In some embodiments, a DNA or RNA ligase is used in the synthesis of the circular polynucleotides. In some embodiments, either the 5′- or 3′-end of the linear polyribonucleotide for circularization can encode a ligase ribozyme sequence such that during in vitro transcription, the resultant linear polyribonucleotide for circularization includes an active ribozyme sequence capable of ligating the 5′-end of the linear polyribonucleotide for circularization to the 3′-end of the linear polyribonucleotide for circularization. The ligase ribozyme may be derived, e.g., from the Group I Intron, Hepatitis Delta Virus, Hairpin ribozyme or may be selected by SELEX (systematic evolution of ligands by exponential enrichment). The ribozyme ligase reaction may take, e.g., 1 to 24 hours at temperatures between 0 and 37° C.

In some embodiments, a linear polyribonucleotide for circularization may include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). In some embodiments, the population of polyribonucleotides including circular polyribonucleotides, linear polyribonucleotides, and linear polydeoxyribonucleotides is contacted with RppH prior to digesting at least a portion of the linear polyribonucleotides with a 5′ exonuclease or a 3′ exonuclease. In some embodiments, the population of polyribonucleotides including circular polyribonucleotides, linear polyribonucleotides, and linear polydeoxyribonucleotides is contacted with T4 Polynucleotide Kinase prior to digesting at least a portion of the linear polyribonucleotide with a 5′ exonuclease or 3′ exonuclease.

Alternately, converting the 5′ triphosphate of the linear polyribonucleotide for circularization into a 5′ monophosphate may occur by a two-step reaction including: (a) contacting the 5′ nucleotide of the linear polyribonucleotide for circularization with a phosphatase (e.g., Antarctic Phosphatase, Shrimp Akaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

In some embodiments, the linear polyribonucleotide for circularization is synthesized using IVT and an RNA polymerase, where the nucleotide mixture used for IVT may contain an excess of guanosine monophosphate relative to guanosine triphosphate to preferentially produce RNA with a 5′ monophosphate; the purified IVT product may be circularized using a splint DNA.

In some embodiments, circularization efficiency of the circularization methods provided herein is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, the circularization method provided has a circularization efficiency of between about 10% and about 100%; for example, the circularization efficiency may be about 15%, about 20%, about 25%, about 30%, about 35%, about 40%, about 45%, about 50%, about 55%, about 60%, about 65%, about 70%, about 75%, about 80%, about 85%, about 90%, about 95%, and about 99%. In some embodiments, following digestion with the 5′ exonuclease and the 3′ exonuclease the percent (w/w) of the circular polyribonucleotides is between 40% and 95% (e.g., between 40% and 90%, 40% and 80%, 40% and 70%, 40% and 60%, 40% and 50%, 50% and 95%, 60% and 95%, 70% and 95%, 80% and 95%, or 90% and 95%) of the total polynucleotides. In some embodiments, following digestion with the 5′ exonuclease and the 3′ exonuclease the percent (w/w) of the circular polyribonucleotides is between 60% and 95% (e.g., between 60% and 90%, 60% and 80%, 60% and 70%, 70% and 95%, 80% and 95%, or 90% and 95%) of the total polynucleotides.

In some embodiments, enzymatic methods of circularization may be used to generate the circular polyribonucleotide. In some embodiments, a ligation enzyme, e.g., DNA or RNA ligase, may be used to generate a template of the circular polyribonuclease or complement, a complementary strand of the circular polyribonuclease, or the circular polyribonuclease.

Circularization of the circular polyribonucleotide may be accomplished by methods known in the art, for example, those described in “RNA circularization strategies in vivo and in vitro” by Petkovic and Muller from Nucleic Acids Res, 2015, 43(4): 2454-2465, and “In vitro circularization of RNA” by Muller and Appel, from RNA Biol, 2017, 14(8):1018-1027.

The circular polyribonucleotide may encode a sequence and/or motif useful for replication. Exemplary replication elements are described in paragraphs [0280]-[0286] of International Patent Publication No. WO2019/118919, which is hereby incorporated by reference in its entirety.

### Detection of Linear and Circular RNA

The presence of linear RNA in pharmaceutical circular RNA preparations can have unexpected and sometimes undesirable effects. Thus, circular RNAs may be enriched, separated, and/or purified relative to linear RNA; methods (e.g., methods of manufacturing circular RNA preparations) whereby linear RNAs can be monitored, evaluated and/or controlled; and methods of using such pharmaceutical compositions and preparations. In some embodiments, a circular RNA preparation has no more than a threshold level of linear RNA, e.g., a circular RNA preparation is enriched over linear RNA or purified to reduce linear RNA. In some embodiments, the circular polyribonucleotide population is enriched such that a mixture of circular polyribonucleotides and linear polyribonucleotides includes a threshold level of circular polyribonucleotides.

Generally, detection and quantitation of an element in a pharmaceutical preparation includes the use of a reference standard that is either the component of interest (e.g., circular RNA, linear RNA, fragment, impurity, etc.) or is a similar material (e.g., using a linear RNA structure of the same sequence as a circular RNA structure as a standard for circular RNA), or includes the use of an internal standard or signal from a test sample. In some embodiments, the standard is used to establish the response from a detector for a known or relative amount of material (response factor). In some embodiments, the response factor is determined from a standard at one or multiple concentrations (e.g., using linear regression analysis). In some embodiments, the response factor is then used to determine the amount of the material of interest from the signal due to that component. In some embodiments, the response factor is a value of one or is assumed to have a value of one.

In some embodiments, detection, and quantification of linear versus circular RNA in the pharmaceutical composition is determined using a comparison to a linear version of the circular polyribonucleotides. In some embodiments, the mass of total ribonucleotide in the pharmaceutical composition is determined using a standard curve generated using a linear version of the circular polyribonucleotide and assuming a response factor of one. In some embodiments, a w/w percentage of circular polyribonucleotide in the pharmaceutical preparation is determined by a comparison of a standard curve generated by band intensities of multiple known amounts of a linear version of the circular polyribonucleotide to a band intensity of the circular polyribonucleotide in the pharmaceutical preparation. In some embodiments, the bands are produced during gel-base electrophoresis, and the band intensities are measured by a gel imager (e.g., an E-gel Imager). In some embodiments, a circular polyribonucleotide preparation includes less than a threshold amount (e.g., where the threshold amount is a reference criterion, e.g., a pharmaceutical release specification for the circular polyribonucleotide preparation) of linear polyribonucleotide molecules when evaluated as described herein.

In some embodiments, detection, and quantification of nicked versus total RNA in the pharmaceutical composition is determined by sequencing after gel extraction of the preparation including the circular RNA. In some embodiments, detection, and quantification of nicked versus linear RNA in the pharmaceutical composition is determined by sequencing after gel extraction of the preparation including the circular RNA. In some embodiments, a circular polyribonucleotide preparation includes less than a threshold amount (e.g., where the threshold amount is a reference criterion, e.g., a pharmaceutical release specification for the circular polyribonucleotide preparation) of nicked RNA, linear RNA, or combined linear and nicked RNA when evaluated as described herein. For example, the reference criterion for the amount of linear polyribonucleotide molecules present in the preparation is no more than 30%, 20%, 15%, 10%, 1%, 0.5%, or 0.1% linear polyribonucleotide molecules, or any percentage therebetween, relative to total ribonucleotide molecules in the preparation. In some embodiments, the reference criterion for the amount of nicked polyribonucleotide molecules present in the preparation is no more than 30%, 20%, 15%, 10%, 1%, 0.5%, or 0.1%, or any percentage therebetween, nicked polyribonucleotide molecules relative to total ribonucleotide molecules in the preparation. In some embodiments, the reference criterion for the amount of linear and nicked polyribonucleotide molecules present in the preparation is no more than 40%, 30%, 20%, 15%, 10%, 1%, 0.5%, or 0.1%, or any percentage therebetween, combined linear polyribonucleotide and nicked polyribonucleotide molecules relative to total ribonucleotide molecules in the preparation.

In some embodiments, the standard is run under the same conditions as the sample. For example, the standard is run with the same type of gel, same buffer, and same exposure as the sample. In further embodiments, the standard is run in parallel with the sample. In some embodiments, a quantification of an element is repeated (e.g., twice or in triplicate) in a plurality of samples from the subject preparation to obtain a mean result. In some embodiments, quantitation of a linear RNA is measured using parallel capillary electrophoresis (e.g., using a Fragment Analyzer or analytical HPLC with UV detection).

Alternatively, to measure the amount of linear and circular RNA present, qPCR reverse transcription (RT-qPCR) using two sets of primers: one across the ligation site and one specific to the ORF. The primers across the ligation site report on the levels of circular RNA whereas the primers specific to the ORF report on both circular and linear RNA. In some embodiments, the reverse transcription reactions are performed with a reverse transcriptase (e.g., Super-Script II: RNase H; Invitrogen) and random hexamers in accordance with the manufacturer's instruction. In some embodiments, the amplified PCR products are analyzed using polyacrylamide gel electrophoresis and visualized by ethidium bromide staining. In some embodiments, to estimate the enrichment factor of the circular polyribonucleotides, the PCR products may be quantified using densitometry, and the concentrations of total RNA samples may be measured by UV absorbance.

### Other Embodiments

Various modifications and variations of the described compositions, methods, and uses of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

All publications, patents, and patent applications are herein incorporated by reference in their entirety to the same extent as if each individual publication, patent, or patent application was specifically and individually indicated to be incorporated by reference in its entirety.

### EXAMPLES

The following examples, which are intended to illustrate, rather than limit, the disclosure, are put forth to provide those of ordinary skill in the art with a description of how the compositions and methods described herein may be used, made, and evaluated. The examples are intended to be purely exemplary of the disclosure and are not intended to limit the scope of what the inventors regard as their invention.

### Example 1: In Vitro Production of Circular RNAs Encoding GFP

This example describes in vitro production of the circular polyribonucleotide.

The circular polyribonucleotide was designed with an IRES and an ORF encoding GFP, and two spacer elements flanking the IRES-ORF. The circular polyribonucleotide was generated using in vitro transcription (IVT). Briefly, unmodified linear RNA was synthesized by in vitro transcription using T7 RNA polymerase from a DNA segment with the above elements. Transcribed RNA was purified with an RNA purification system (New England Biolabs, Inc.), treated with RNA 5′phosphohydrolase (RppH) (New England Biolabs, M0356) following the manufacturer's instructions, and purified again with the RNA purification system. RppH-treated linear RNA was circularized using a splint DNA.

Circular polyribonucleotide was generated by splint-ligation as follows: Transcribed linear polyribonucleotide and a DNA splint (5′-CAATCGACGGTCCCCCTAGAAGATATGCTG-3′; SEQ ID NO: 1) were mixed and annealed and treated with an RNA ligase to produce a post-ligation polyribonucleotide mixture.

### Example 2: Digestion of Linear RNA with 5′ Exonuclease Over Time

This Example Demonstrates that a 5′ Exonuclease can be Used to Degrade Linear Polyribonucleotide.

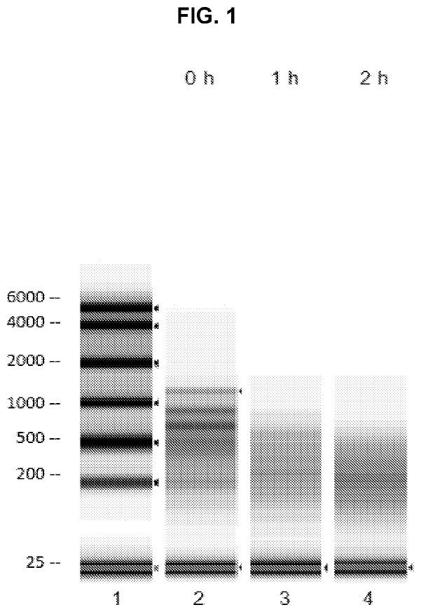

A by-product of the circularization reaction is unreacted linear polyribonucleotide. To remove the unreacted linear polyribonucleotide after the circularization reaction, a single-stranded 5′ exonuclease, Xrn-1, was used to degrade the non-circularized linear RNA. To understand the kinetics of Xm-1 digestion, 1 μg of the post-ligation polyribonucleotide mixture including both circular and linear polyribonucleotides produced as described in Example 1 was digested with 1 U (1 μl) Xrn-1 (New England Biolabs) in 1×NEB3 buffer (New England Biolabs) where the total reaction volume was 10 μL. The reaction was monitored over time for completion; at each time point, a fraction of the Xrn-1 digestion was removed and then quenched using 25 mM EDTA. The polyribonucleotide mixture was digested for either 0, 1, or 2 hours before the reaction was quenched, and then the digestion was separated and visualized using capillary electrophoresis on the 4150 Tapestation System with RNA ScreenTape (Agilent) according to the manufacturer's instructions (FIG. 1). The zero time point (lane 2) showed some digestion as the Xrn-1 enzyme was not fully quenched prior to the addition of the nucleic acid. After one hour, the unreacted linear RNA had undergone appreciable degradation and is no longer full-length, ˜2.5 kb in size (FIG. 1).

### Example 3: Removal of Annealed DNA Splint after Circularization is Required Prior to Enzymatic Removal of Linear RNA by an Exonuclease

This example demonstrates that the presence of a DNA splint prevented the digestion of linear polyribonucleotide by exonuclease.

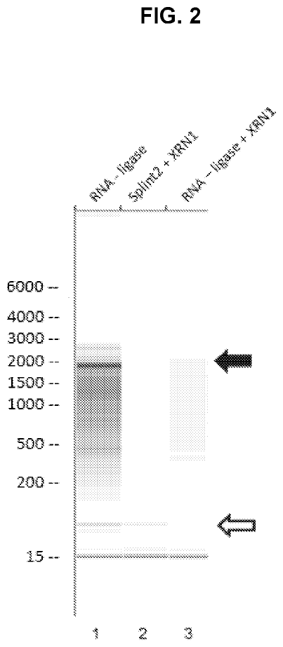

A mock circularization reaction (circularization without ligase) was performed by the combination of 100 pmol of RppH-treated linear in vitro transcribed RNA produced as described in Example 1 with 2 μM splint DNA in 1×T4 RNA Ligase II buffer (New England Biolabs) and incubated at 75° C. for 10 min with a final reaction volume of 97 μL in the absence of an RNA ligase. The mixture was then cooled to room temperature for 20 min at which point 40 U of Murine RNase inhibitor (New England Biolabs) was added to the reaction and the volume was brought up to 100 μL. The samples were incubated while shaking at 37° C. for 4 h followed by increasing the temperature to 80° C. for 5 min. After the mock circularization, the sample was extracted with phenol:chloroform:iso-amyl alcohol (2524:1) (v/v/v), Biotechnology Grade 1 Phase (VWR International), ethanol precipitated, and quantified. The purified RNA (5 μg) or DNA splint (control, 20 pmol) was reacted with 5 U of Xrn-1 (New England Biolabs) in 1×NEB3 buffer with 0.4 U/μl RNase inhibitor where the final reaction volume was 100 μL. After 1 h incubation at 37° C., samples were separated and visualized by capillary electrophoresis on the 5300 fragment analyzer using the DNF-471 RNA kit (15 NT) according to manufacturer's instructions (Agilent) (FIG. 2). In comparison to lanes 3 and 4 in FIG. 1, lane 3 in FIG. 2 demonstrates that the annealing step allows the splint DNA (open arrow) to partially protect linear polyribonucleotide (black arrow) from digestion by a single stranded exonuclease.

### Example 4: Enrichment of Circular RNA after Splint Ligation Using DNase I and 5′ Exonuclease

This example demonstrates that circular RNA after splint ligation was enriched using a combination of a DNase I and 5′ exonuclease.

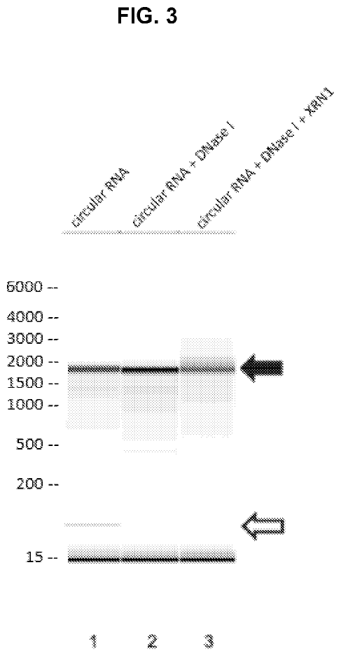

To improve the removal of linear polyribonucleotides, DNase I was added prior to exonuclease digestion to remove the splint DNA. Briefly, RppH-treated linear RNA produced as described in Example 1 was circularized by incubating 200 pmol RNA with 2 μM splint DNA and 1×T4 RNA Ligase II buffer at 75° C. for 10 min where the final volume was 194 μL. The mixture was cooled to room temperature for 20 min followed by the addition of 4 μL ligase and 2 μL RNase inhibitor (80 U). The samples were incubated at 37° C. for 4 h while shaking at 300 rpm and after incubation ethanol precipitated to obtain a post-ligation polyribonucleotide mixture. The post-ligation polyribonucleotide mixture (500 ng) was reacted with 0.1 μL DNase 1 (0.2 U; New England Biolabs), 0.3 μL (12 U) RNase inhibitor and 1× DNase I buffer in a final volume of 25 μL. Samples were incubated at 37° C. for 30 min, quenched with 4 mM EDTA and heat inactivated at 75° C. for 10 min followed by an ethanol precipitation that was resuspend in 22 μl water. The sample (20 μL) was combined with 0.5 μl Xrn-1 and 0.3 μl (12 U) RNase inhibitor in 1×NEB3 buffer to a final volume of 25 μL and incubated at 37° C. for 1 h. The reaction was then extracted with phenol:chloroform:iso-amyl alcohol, ethanol precipitated, and subsequently analyzed by capillary electrophoresis using an Agilent 5300 fragment analyzer using the DNF-471 RNA kit (15 NT) (FIG. 3) according to manufacturer's instructions. The RNA successfully circularized and was not digested by Xm-1 after DNase I digestion (FIG. 3; black arrow in lane 3).

### Example 5: Enzymatic Purification of Circularized RNA Improves Yield Over Gel Extraction

This example demonstrates that enzymatic purification of circularized RNA improved yield over gel extraction.

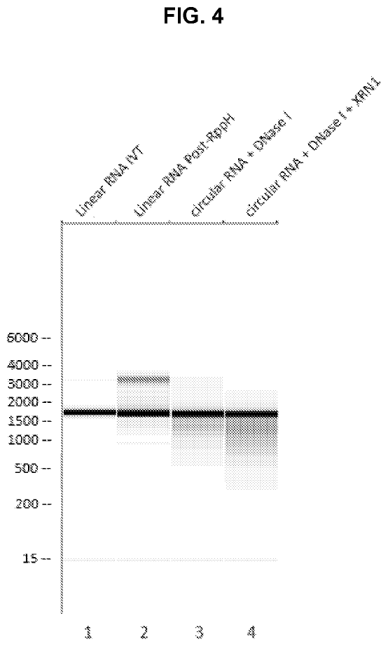

Enzymatic purification was compared to that of the previously described gel purification of circularized RNA. A scaled-up circularization reaction (200 or 500 pmol RNA) was performed as described above where at the completion of the reaction calcium chloride (0.5 mM final concentration) and 30 μL DNase I was added. The reaction was incubated at 37° C. for 10 min. The DNase I reaction was then quenched by 5 mM EDTA (final concentration), and the reaction was further heat inactivated at 65° C. followed by a phenol:choroform:iso-amyl alcohol extraction and ethanol precipitation. The RNA was quantified and then 200 μg of RNA was digested with 40 μl Xrn-1 in a reaction that contained 2 μl RNase inhibitor and 1×NEB3 buffer with the total volume of 200 μl. The sample was incubated for 6 hours at 37° C. and subsequently extracted with phenol:chloroform:iso-amyl alcohol and ethanol precipitated into a final volume of 50 μl water. The resulting RNA and intermediate RNAs were separated and visualized by capillary electrophoresis on the Agilent 5300 fragment analyzer using the DNF-471 RNA kit (15 NT) according to manufacturer's instructions (FIG. 4).

The yield of circularized RNAs purified either using the previously described gel extraction or the enzymatic method described above were analyzed using a NanoDrop™ One Microvolume UV-Vis Spectrophotometer (ThermoFisher Scientific). The yields were compared as a percentage of the amount of RNA that went into the gel electrophoresis purification or enzymatic purification of linear RNA after circularization. After linear RNA removal and the remaining circular RNA being extracted with phenol:chloroform:iso-amyl alcohol and ethanol precipitated, the enzymatic method resulted in a ˜30-fold increase in yield and improved purity (Table 2).

TABLE 2
Yield and purity comparison of both
methods of circular RNA isolation

| Sample | Nucleic Acid | A₂₆₀/ | A₂₆₀/ | Yield from | Total Yield |
|---|---|---|---|---|---|
| Name | (ng/μL) | A₂₈₀ | A₂₃₀ | last step (%) | (μg) |
| Gel | 21.864 | 1.921 | 1.477 | 2 | 1.1 |
| purification | | | | | |
| method | | | | | |
| Enzymatic | 230.351 | 2.100 | 2.509 | 58 | 115.2 |
| method | | | | | |

### Example 6: Enrichment of Circular RNA after Splint Ligation Using DNase I and a 3′ Exonuclease

This example describes enrichment of circular RNA after splint ligation using a combination of DNase I and 3′ exonuclease.

The method of enzymatic purification of circular RNA takes advantage of the property that linear RNA has free ends that are susceptible to exonucleases whereas circularized RNA is protected from exonucleases as it has no free ends. To this end, an alternative to 5′ exonucleases, such as Xrn-1 described above, are 3′ exonucleases such as RNase R.

In some embodiments, after the ligation reaction to circularize RNA, the reaction is treated with DNase I followed by exonuclease treatment with RNase R via incubation in RNase R reaction buffer at 37° C. for 10 min using 1 U/μg RNA. The concentration of RNase R may be lowered and combined with extended incubation times. After treatment with RNase R 3′ exonuclease, the reaction is heat inactivated at 65° C. for 20 min and then purified using phenol:chloroform:iso-amyl alcohol extraction and ethanol precipitation.

1. A method of producing an enriched population of circular polyribonucleotides, the method comprising:
(a) providing a splint ligation reaction product comprising circular polyribonucleotides, linear polyribonucleotides, and linear polydeoxyribonucleotides;
(b) reacting the splint ligation reaction product of step (a) with DNase I, wherein DNase I digests at least a portion of the linear polydeoxyribonucleotides to produce a first digested mixture;
(c) reacting the first digested mixture of step (b) with an exonuclease, wherein the exonuclease digests at least a portion of the linear polyribonucleotides to produce a second digested mixture;
wherein the second digested mixture comprises an enriched population of circular polyribonucleotides.

2. The method of claim 1, wherein DNase I of digesting step (b) is in an amount of between 0.1 U/μg and 1 U/μg.

3. The method of claim 1 or claim 2, wherein digesting step (b) is performed for at least 10 minutes.

4. The method of any one of claim 1-3, wherein digesting step (b) is performed at a temperature of about 37° C.

5. The method of any one of claims 1-4, wherein the exonuclease of digesting step (c) is a 5′ exonuclease.

6. The method of claim 5, wherein the 5′ exonuclease of digesting step (c) is a 5′-phosphate dependent exonuclease.

7. The method of claim 5, wherein the 5′ exonuclease is Xrn-1.

8. The method of claim 7, wherein the Xrn-1 is in an amount of between 0.1 U/μg and 1 U/μg.

9. The method of any one of claims 1-4, wherein the exonuclease of digesting step (c) is a 3′ exonuclease.

10. The method of claim 9, wherein the 3′ exonuclease is Exonuclease T.

11. The method of any one of claims 1-10, wherein digesting step (c) is performed for at least 1 hour.

12. The method of any one of claims 1-11, wherein digesting step (c) is performed at a temperature of about 37° C.

13. A method of producing an enriched population of circular polyribonucleotides, the method comprising:
(a) providing a linear polyribonucleotide having a 5′ and 3′ end and a polydeoxyribonucleotide that has a first region that hybridizes to the 5′ end of the linear polyribonucleotide and a second region that hybridizes to the 3′ end of the linear polyribonucleotide,
(b) ligating the 5′ end of the linear polyribonucleotide to the 3′ end of the linear polyribonucleotide to produce a splint ligation reaction product comprising circular polyribonucleotides, linear polyribonucleotides, and linear polydeoxyribonucleotides;
(c) reacting the splint ligation reaction product of step (b) with DNase I, wherein the DNase I digests at least a portion of the polydeoxyribonucleotide to produce a first digested mixture;
(d) reacting the first digested mixture of step (c) with an exonuclease, wherein the exonuclease digests at least a portion of the linear polyribonucleotide to produce a second digested mixture,
wherein the second digested mixture comprises an enriched population of circular polyribonucleotides.

14. The method of claim 13, wherein DNase I of digesting step (c) is in an amount of between 0.1 U/μg and 1 U/μg.

15. The method of claim 13 or claim 14, wherein digesting step (c) is performed for at least 10 minutes.

16. The method of any one of claim 13-15, wherein digesting step (c) is performed at a temperature of about 37° C.

17. The method of any one of claims 13-16, wherein the exonuclease of digesting step (d) is a 5′ exonuclease.

18. The method of claim 17, wherein the 5′ exonuclease of digesting step (d) is a 5′-phosphate dependent exonuclease.

19. The method of claim 18, wherein the 5′ exonuclease is Xrn-1.

20. The method of claim 19, wherein the Xrn-1 is in an amount of between 0.1 U/μg and 1 U/μg.

21. The method of any one of claims 13-16, wherein the exonuclease of digesting step (d) is a 3′ exonuclease.

22. The method of claim 21, wherein the 3′ exonuclease is Exonuclease T.

23. The method of any one of claims 13-22, wherein digesting step (d) is performed for at least 1 hour.

24. The method of any one of claims 13-23, wherein digesting step (d) is performed at a temperature of about 37° C.


SEQUENCE LISTING (23 sequences)

SEQ ID NO: 1 - DNA, 30 bp, Artificial Sequence
  Synthetic Construct
caatcgacggtccccctagaagatatgctg

SEQ ID NO: 2 - protein, 8 aa, Artificial Sequence
  Synthetic Construct
  MISC_FEATURE (2)..(2): Xaa is Val or Ile
  MISC_FEATURE (4)..(4): Xaa is any amino acid
DXEXNPGP

SEQ ID NO: 3 - protein, 9 aa, Artificial Sequence
  Synthetic Construct
GDVESNPGP

SEQ ID NO: 4 - protein, 9 aa, Artificial Sequence
  Synthetic Construct
GDIEENPGP

SEQ ID NO: 5 - protein, 7 aa, Artificial Sequence
  Synthetic Construct
VEPNPGP

SEQ ID NO: 6 - protein, 7 aa, Artificial Sequence
  Synthetic Construct
IETNPGP

SEQ ID NO: 7 - protein, 9 aa, Artificial Sequence
  Synthetic Construct
GDIESNPGP

SEQ ID NO: 8 - protein, 9 aa, Artificial Sequence
  Synthetic Construct
GDVELNPGP

SEQ ID NO: 9 - protein, 9 aa, Artificial Sequence
  Synthetic Construct
GDIETNPGP

SEQ ID NO: 10 - protein, 8 aa, Artificial Sequence
  Synthetic Construct
GDVENPGP

SEQ ID NO: 11 - protein, 9 aa, Artificial Sequence
  Synthetic Construct
GDVEENPGP

SEQ ID NO: 12 - protein, 9 aa, Artificial Sequence
  Synthetic Construct
GDVEQNPGP

SEQ ID NO: 13 - protein, 7 aa, Artificial Sequence
  Synthetic Construct
IESNPGP

SEQ ID NO: 14 - protein, 9 aa, Artificial Sequence
  Synthetic Construct
GDIELNPGP

SEQ ID NO: 15 - protein, 9 aa, Artificial Sequence
  Synthetic Construct
HDIETNPGP

SEQ ID NO: 16 - protein, 9 aa, Artificial Sequence
  Synthetic Construct
HDVETNPGP

SEQ ID NO: 17 - protein, 9 aa, Artificial Sequence
  Synthetic Construct
HDVEMNPGP

SEQ ID NO: 18 - protein, 9 aa, Artificial Sequence
  Synthetic Construct
GDMESNPGP

SEQ ID NO: 19 - protein, 9 aa, Artificial Sequence
  Synthetic Construct
GDVETNPGP

SEQ ID NO: 20 - protein, 9 aa, Artificial Sequence
  Synthetic Construct
GDIEQNPGP

SEQ ID NO: 21 - protein, 8 aa, Artificial Sequence
  Synthetic Construct
DSEFNPGP

SEQ ID NO: 22 - protein, 9 aa, Artificial Sequence
  Synthetic Construct
  MISC_FEATURE (1)..(1): Xaa is absent or Gly or His
  MISC_FEATURE (2)..(2): Xaa is absent or Asp or Gly
  MISC_FEATURE (3)..(3): Xaa is Asp or Val or Ile or Ser or Met
  MISC_FEATURE (5)..(5): Xaa is any amino acid
XXXEXNPGP

SEQ ID NO: 23 - RNA, 9 nt, Artificial Sequence
  Synthetic Construct
  misc_feature (3)..(3): r is a or g
ccrccaugg